SEQUENCE LISTING (32 sequences)

SEQ ID NO: 1 - DNA, 15 bp, synthetic construct
  source 1..15: mol_type = other DNA
cacaagcctctcaca

SEQ ID NO: 2 - DNA, 61 bp, synthetic construct
  source 1..61: mol_type = other DNA
gggccttttggccctgtgagaggcttgtgaatactccgtaacagtaacagccgctgtggat

SEQ ID NO: 3 - DNA, 15 bp, synthetic construct
  source 1..15: mol_type = other DNA
cacagactacccaca

SEQ ID NO: 4 - DNA, 61 bp, synthetic construct
  source 1..61: mol_type = other DNA
gggccttttggccctgtgggtagtctgtgtcactggtaatctggttgccatgtagagcgac

SEQ ID NO: 5 - DNA, 15 bp, synthetic construct
  source 1..15: mol_type = other DNA
cacagccaatccaca

SEQ ID NO: 6 - DNA, 61 bp, synthetic construct
  source 1..61: mol_type = other DNA
gggccttttggccctgtggattggctgtggatggcctagatgtgtcagctactgacagagc

SEQ ID NO: 7 - DNA, 15 bp, synthetic construct
  source 1..15: mol_type = other DNA
cacatcgctctcaca

SEQ ID NO: 8 - DNA, 61 bp, synthetic construct
  source 1..61: mol_type = other DNA
gggccttttggccctgtgagagcgatgtgtacagaagaaaatgagatgcaactcagttcaa

SEQ ID NO: 9 - DNA, 15 bp, synthetic construct
  source 1..15: mol_type = other DNA
cacattgccaccaca

SEQ ID NO: 10 - DNA, 61 bp, synthetic construct
  source 1..61: mol_type = other DNA
gggccttttggccctgtggtggcaatgtgataacaaccaaaagggaacagtgagtagaaaa

SEQ ID NO: 11 - DNA, 15 bp, synthetic construct
  source 1..15: mol_type = other DNA
cacatcaagcccaca

SEQ ID NO: 12 - DNA, 61 bp, synthetic construct
  source 1..61: mol_type = other DNA
gggccttttggccctgtgggcttgatgtgtctctatcttgaaccttcttcataagagggag

SEQ ID NO: 13 - DNA, 44 bp, synthetic construct
  source 1..44: mol_type = other DNA
gggccttttggccctgtgagaggcttgtgtgtgagaggcttgtg

SEQ ID NO: 14 - DNA, 44 bp, synthetic construct
  source 1..44: mol_type = other DNA
gggccttttggccctgtgggtagtctgtgtgtgggtagtctgtg

SEQ ID NO: 15 - DNA, 44 bp, synthetic construct
  source 1..44: mol_type = other DNA
gggccttttggccctgtggattggctgtgtgtggattggctgtg

SEQ ID NO: 16 - DNA, 44 bp, synthetic construct
  source 1..44: mol_type = other DNA
gggccttttggccctgtgagagcgatgtgtgtgagagcgatgtg

SEQ ID NO: 17 - DNA, 44 bp, synthetic construct
  source 1..44: mol_type = other DNA
gggccttttggccctgtggtggcaatgtgtgtggtggcaatgtg

SEQ ID NO: 18 - DNA, 44 bp, synthetic construct
  source 1..44: mol_type = other DNA
gggccttttggccctgtgggcttgatgtgtgtgggcttgatgtg

SEQ ID NO: 19 - DNA, 66 bp, synthetic construct
  source 1..66: mol_type = other DNA
acatcatcatgggccttttggcccatgatgatgtaatactccgtaacagtaacagccgctgtggat

SEQ ID NO: 20 - DNA, 66 bp, synthetic construct
  source 1..66: mol_type = other DNA
accaataatagggccttttggccctattattggttcactggtaatctggttgccatgtagagcgac

SEQ ID NO: 21 - DNA, 66 bp, synthetic construct
  source 1..66: mol_type = other DNA
aataaacctagggccttttggccctaggtttattgatggcctagatgtgtcagctactgacagagc

SEQ ID NO: 22 - DNA, 66 bp, synthetic construct
  source 1..66: mol_type = other DNA
aaatactctcgggccttttggcccgagagtattttacagaagaaaatgagatgcaactcagttcaa

SEQ ID NO: 23 - DNA, 66 bp, synthetic construct
  source 1..66: mol_type = other DNA
attattcactgggccttttggcccagtgaataatataacaaccaaaagggaacagtgagtagaaaa

SEQ ID NO: 24 - DNA, 66 bp, synthetic construct
  source 1..66: mol_type = other DNA
actttttttcgggccttttggcccgaaaaaaagttctctatcttgaaccttcttcataagagggag

SEQ ID NO: 25 - DNA, 44 bp, synthetic construct
  source 1..44: mol_type = other DNA
acatcatcatgggccttttggcccatgatgatgtatgatgatgt

SEQ ID NO: 26 - DNA, 44 bp, synthetic construct
  source 1..44: mol_type = other DNA
accaataatagggccttttggccctattattggttattattggt

SEQ ID NO: 27 - DNA, 44 bp, synthetic construct
  source 1..44: mol_type = other DNA
aataaacctagggccttttggccctaggtttatttaggtttatt

SEQ ID NO: 28 - DNA, 44 bp, synthetic construct
  source 1..44: mol_type = other DNA
aaatactctcgggccttttggcccgagagtatttgagagtattt

SEQ ID NO: 29 - DNA, 44 bp, synthetic construct
  source 1..44: mol_type = other DNA
attattcactgggccttttggcccagtgaataatagtgaataat

SEQ ID NO: 30 - DNA, 44 bp, synthetic construct
  source 1..44: mol_type = other DNA
actttttttcgggccttttggcccgaaaaaaagtgaaaaaaagt

SEQ ID NO: 31 - protein, 6 aa, synthetic construct
HHHHHH

SEQ ID NO: 32 - DNA, 14 bp, synthetic construct
  source 1..14: mol_type = other DNA
gggccttttggccc

(19) United States
(12) Patent Application Publication
(10) Pub. No.: US 2026/0103742 A1
(43) Pub. Date: Apr. 16, 2026

(54) METHODS AND SYSTEMS FOR TARGET EXTENDED AMPLIFICATION

(71) Applicant: 10x Genomics, Inc., Pleasanton, CA (US)

(72) Inventor: Su WANG, Berkeley, CA (US)

(21) Appl. No.: 19/358,316

(22) Filed: Oct. 14, 2025

Related U.S. Application Data
(60) Provisional application No. 63/707,623, filed on Oct. 15, 2024.

(51) Int. Cl. C12Q 1/6806; C12Q 1/6874

(52) U.S. Cl. CPC C12Q 1/6806; C12Q 1/6874

(57) ABSTRACT

The present disclosure relates in some aspects to methods, systems, and kits for analyzing a biological sample comprising performing extension of a target ribonucleic acid (RNA). In some aspects, RNA cutting enzyme and/or a nucleic acid oligonucleotide are used to generate a free 3′ end of the target RNA for an extension reaction.

### CROSS-REFERENCE TO RELATED APPLICATIONS

This application claims priority to U.S. Provisional Patent Application No. 63/707,623, filed Oct. 15, 2024, entitled “METHODS AND SYSTEMS FOR TARGET EXTENDED AMPLIFICATION”, which is herein incorporated by reference in its entirety for all purposes.

### SEQUENCE LISTING

The instant application contains a Sequence Listing which has been submitted electronically in XML format and is hereby incorporated by reference in its entirety. Said XML copy, created on Oct. 7, 2025, is named 43487-4043_201_SL.xml and is 29,719 bytes in size.

### FIELD

The present disclosure relates in some aspects to methods for in situ analysis of target nucleic acids in a biological sample. In some aspects, the methods and compositions provided herein provides improved signal amplification for detecting analytes in a sample.

### BACKGROUND

Methods are available for analyzing nucleic acids in a biological sample in situ, such as in a cell or a tissue sample. For instance, advances in single molecule fluorescent hybridization (smFISH) have enabled nanoscale-resolution imaging of RNA in cells and tissues. Amplification methods such as rolling circle amplification (RCA)-based detection methods allow detection of target nucleic acids such as RNA in cells and tissues. However, in some cases RCA-based assay methods for in situ analysis may suffer from some drawbacks. Improved methods for generating an amplification product for sequence detection, e.g., in situ analysis, are needed. The present disclosure addresses these and other needs.

### SUMMARY

There are various ways to generate an amplification product for downstream detection in biological samples (e.g., at their relative spatial locations in situ). However, certain ways of generating the amplification product may require a complex workflow that is not suitable for automation. For example, one method for generating an amplification product is using rolling circle amplification (RCA) of a circular template. In some aspects, the RCA workflow requires that a probe hybridize to a target nucleic acid, followed by a ligation reaction and an amplification reaction. Due to the various operations required, a library preparation workflow involving RCA may have to be performed manually rather than being automated and performed on an instrument. There is a need for new and improved methods for library preparation that is high throughput and allows for generating amplified sequences for detection.

In some aspects, provided herein is a method of nucleic acid processing, comprising: (a) hybridizing an extension probe to a target ribonucleic acid (RNA), wherein the extension probe comprises (i) a target recognition sequence complementary to a target sequence at the 3′ end of the target RNA, and (ii) a detection sequence; (b) extending the target RNA with a polymerase using the extension probe as a template, thereby generating an extended nucleic acid molecule; and (c) performing further extension of the extended nucleic acid molecule using a plurality of additional extension probes, wherein an additional extension probe of the plurality of additional extension probe comprises (i) at least a portion of the detection sequence of the extension probe or a complement thereof, and (ii) an amplification sequence; thereby generating an extended concatemer comprising the target RNA, the detection sequence or a complement thereof and the amplification sequence or a complement thereof. In some aspects, the method comprises prior to hybridizing the extension probe to the target RNA, cleaving the target RNA. In some instances, the cleavage is 3′ to the target sequence in the target RNA.

In other aspects, provided herein is a method of nucleic acid processing, comprising: (a) generating a cleaved target RNA from a target RNA; (b) hybridizing an extension probe to the cleaved target RNA, wherein the extension probe comprises (i) a target recognition sequence complementary to a target sequence in the target RNA, and (ii) a detection sequence; and (c) extending the cleaved target RNA with a polymerase using the extension probe as a template, thereby generating an extended nucleic acid molecule.

In some embodiments, the generating of the cleaved target RNA (e.g., in (a)) comprises hybridizing a deoxyribonucleic acid (DNA) oligonucleotide to an oligonucleotide hybridization region in the target RNA and cleaving the target RNA in the oligonucleotide hybridization region, thereby generating a cleaved target RNA. In some embodiments, the DNA oligonucleotide is a DNAzyme. In some embodiments, the oligonucleotide hybridization region is adjacent to the 3′ end of the target sequence or is overlapping with the 3′ end of the target sequence.

In some embodiments, the generating of the cleaved target RNA (e.g., in (a)) comprises contacting the target RNA with a complex comprising a guide nucleic acid and an RNA-cutting enzyme to guide cutting of a guide target sequence in the target RNA by the RNA-cutting enzyme, thereby generating the cleaved target RNA.

In some embodiments, the detection sequence is a barcode sequence that identifies the target RNA.

In some embodiments, the method further comprises (d) contacting the extended nucleic acid molecule with a plurality of additional extension probes, wherein an additional extension probe of the plurality of additional extension probes comprises (i) at least a portion of the detection sequence of the extension probe or a complement thereof, and (ii) an amplification sequence; and (e) extending the extended nucleic acid molecule using an additional extension probe of the plurality of additional extension probes as template, thereby generating an extended concatemer. In some embodiments, wherein the amplification sequence is the same sequence as the detection sequence. In some embodiments, the amplification sequence is different from the detection sequence. In some embodiments, the additional extension probe comprises two or more copies of the detection sequence of the extension probe or a complement thereof.

In some embodiments, the plurality of additional extension probes comprises a first additional extension probe and a second extension probe, wherein the first additional extension probe and the second extension probe comprises different amplification sequences.

In some embodiments, the method further comprises detecting the detection sequence and/or a complement thereof.

In some embodiments, the method further comprises detecting the amplification sequence and/or a complement thereof.

In some embodiments, the detection sequence comprises a barcode sequence that identifies the target RNA.

In some embodiments, the amplification sequence comprises a barcode sequence that identifies the target RNA. In some embodiments, the barcode sequence comprises two or more barcode subunits.

In some embodiments, the extended concatemer is not cleaved prior to detecting the detection sequence and/or a complement thereof. In some embodiments, the extended concatemer is not cleaved prior to detecting the amplification sequence and/or a complement thereof.

In some embodiments, the extended nucleic acid molecule comprises the detection sequence or a complement thereof and at least two copies of the amplification sequence or a complement thereof.

In some embodiments, the extension probe comprises a stem loop structure.

In some embodiments, the additional extension probe comprises a stem loop structure.

In some embodiments, the extension probe comprises a stopper molecule or a stopper modification.

In some embodiments, the additional extension probe comprises a stopper molecule or a stopper modification. In some embodiments, the stopper molecule or the stopper modification prevents extension of the extended nucleic acid molecule. In some embodiments, the stopper molecule or the stopper modification is located in the loop structure of the extension probe. In some embodiments, the stopper molecule or the stopper modification is located in the loop structure of the additional extension probe. In some embodiments, the stopper molecule or the stopper modification is located at the end of a stem structure of the extension probe. In some embodiments, the stopper molecule or the stopper modification is located at the end of a stem structure of the additional extension probe.

In some embodiments, the stopper molecule or stopper modification comprises a chemical modification. In some embodiments, the chemical modification is a phosphoramidite. In some embodiments, the extension probe comprises a C3 spacer phosphoramidite. In some embodiments, the chemical modification is a sulfhydryl reactive group. In some embodiments, the chemical modification is an azide modification configured to be covalently linked an alkyne through click chemistry. In some embodiments, the chemical modification is a dibenzocyclooctyne (DBCO) group or a bicyclononyne (BCN) group. In some embodiments, the chemical modification is an amine or a carboxyl group.

In some embodiments, the stopper molecule or stopper modification is selected from a triethylene glycol (TEG), 18-atom hexa-ethylene glycol, adenylation, digoxigenin, cholesteryl-TEG, and 3-cyanovinylcarbazole (CNVK).

In some embodiments, the extension probe and/or the additional extension probe comprises a synthetic non-DNA linker configured to terminate polymerization, optionally wherein the synthetic non-DNA linker comprises iso-dG or iso-dC.

In some embodiments, the stopper molecule is a first nucleotide and the extension and/or further extension is performed with a plurality of free nucleotides that lack a free nucleotide that base-pairs with the first nucleotide. In some embodiments, the stopper molecule is guanine, and the extension is performed with a plurality of free nucleotides that lacks cytosine. In some embodiments, the stopper molecule is cytosine, and the extension is performed with a plurality of free nucleotides that lacks guanine. In some embodiments, the stopper molecule is thymine, and the extension is performed with a plurality of free nucleotides that lacks adenine. In some embodiments, the stopper molecule is adenine, and the extension is performed with a plurality of free nucleotides that lacks thymine.

In some embodiments, the stopper molecule is a first nucleotide and the extension and/or further extension is performed with a plurality of free nucleotides comprising a terminating nucleotide that base pairs with the first nucleotide. In some embodiments, the terminating nucleotide is a reversible terminator nucleotide comprising an azidomethyl group, an amino group, a nitrobenzyl group, an allyl group, a carbonate, a functionalized photocleavable ether, a methyl group, or a cyanoethyl group. In some embodiments, the terminating nucleotide is an irreversible terminator nucleotide, wherein the irreversible terminator nucleotide is a 3′ dideoxynucleotide. In some embodiments, the stopper molecule is guanine, and the extension is performed with a plurality of free nucleotides comprising a terminating cytosine. In some embodiments, the stopper molecule is cytosine, and the extension is performed with a plurality of free nucleotides comprising a terminating guanine. In some embodiments, the stopper molecule is thymine, and the extension is performed with a plurality of free nucleotides comprising a terminating adenine. In some embodiments, the stopper molecule is adenine, and the extension is performed with a plurality of free nucleotides comprising a terminating thymine.

In some embodiments, the extension probe comprises a stem loop structure, wherein a loop sequence of the loop of the structure comprises a plurality of the first nucleotide and a stem sequence of the stem loop structure is made of a plurality of nucleotides that is not the first nucleotide.

In some embodiments, the extension probe comprises a loop domain between the detection sequence and a complement of the detection sequence.

In some embodiments, the additional extension probe comprises a loop domain between the amplification sequence and a complement of the amplification sequence.

In some embodiments, the extension probe comprises from 5′ to 3′: the complement of the detection sequence-the loop domain-the detection sequence-the target recognition sequence.

In some embodiments, the additional extension probe comprises from 5′ to 3′: a complement of the amplification sequence-the loop domain-the amplification sequence-the detection sequence or a complement thereof.

In some embodiments, the extension probe comprises at least two different detection sequences.

In some embodiments, the additional extension probe comprises at least two different amplification sequences.

In some embodiments, the extension probe is a linear oligonucleotide.

In some embodiments, the additional extension probe is a linear oligonucleotide. In some embodiments, the extension probe comprises from 3′ to 5′: the detection sequence-an amplification sequence-a first adapter sequence. In some embodiments, the additional extension probe comprises from 3′ to 5′: the first adapter sequence-an additional amplification sequence-a second adapter sequence. In some embodiments, the amplification sequence and the additional amplification sequence are different. In some embodiments, the amplification sequence and the additional amplification sequence are the same. In some embodiments, the first adapter sequence and the second adapter sequence are different. In some embodiments, the first adapter sequence and the second adapter sequence are the same.

In some embodiments, the amplification sequence of the additional extension probe has the same sequence as the detection sequence of the extension probe. In some embodiments, the additional extension probe has the same sequence as the extension probe. In some embodiments, the extension probe is used as the additional extension probe for additional rounds of extension to generate the extended concatemer. In some embodiments, the extension probe and the additional extension probe comprises from 3′ to 5′: the target recognition sequence-the detection sequence-optionally, a loop domain-a complement of the detection sequence-at least a portion of a complement of the target recognition sequence or a portion thereof.

In some embodiments, the target RNA is cleaved using an RNase H. In some embodiments, the RNase H is an RNase H1 or an RNase H2. In some embodiments, the target RNA is contacted with the DNA oligonucleotide and with the RNase H at the same time as providing the extension probe (e.g., in (c)). In some embodiments, the target RNA is contacted with the DNA oligonucleotide and with the RNase H prior to providing the extension probe (e.g., in (c)).

In some embodiments, the target RNA is cleaved prior to providing the extension probe in (c). In some embodiments, the method comprises performing a wash after cleaving the target RNA and before (c). In some embodiments, the oligonucleotide hybridization region and the target sequence overlap by about 1 to about 20 nucleotides. In some embodiments, the oligonucleotide hybridization region and the target sequence overlap by about 8 to about 10 nucleotides. In some embodiments, the oligonucleotide hybridization region and the target sequence overlap by at least 8 nucleotides, at least 9 nucleotides, or at least 10 nucleotides. In some embodiments, the oligonucleotide hybridization region and the target sequence overlap by no more than 15 nucleotides, no more than 12 nucleotides, or no more than 10 nucleotides. In some embodiments, the oligonucleotide hybridization region is about 10 to about 20 nucleotides in length, or about 15 to about 20 nucleotides in length. In some embodiments, the DNA oligonucleotide is about 10 to about 20 nucleotides in length, or about 15 to about 20 nucleotides in length.

In some embodiments, the DNA oligonucleotide is single-stranded.

In some embodiments, the extended nucleic acid molecule or extended concatemer comprises at least 20 nucleotides, at least 30 nucleotides, or at least 40 nucleotides of the target RNA.

In some embodiments, the guide nucleic acid and the RNA-cutting enzyme are bound in the complex before contacting the biological sample. In some embodiments, the guide nucleic acid and the RNA-cutting enzyme are contacted with the biological sample sequentially or simultaneously, and wherein the guide nucleic acid and the RNA-cutting enzyme form the complex in the biological sample.

In some embodiments, the RNA-cutting enzyme is an Argonaute protein. In some embodiments, the Argonaute protein is an RNA-guided Argonaute, and the guide nucleic acid is an RNA molecule. In some embodiments, the Argonaute protein is a eukaryotic Argonaute protein. In some embodiments, the Argonaute protein is Ago2, optionally wherein the Ago2 is Drosophila Ago2. In some embodiments, the Argonaute protein is a DNA-guided Argonaute, and the guide nucleic acid is a DNA molecule. In some embodiments, the Argonaute protein is a prokaryotic Argonaute protein. In some embodiments, the Argonaute protein is a Drosophila Argonaute protein expressed in a mammalian cell line and loaded with the guide nucleic acid prior to a).

In some embodiments, the RNA-cutting enzyme is a CRISPR effector protein and the guide nucleic acid is a CRISPR guide RNA comprising a spacer sequence, wherein the spacer sequence hybridizes to the target RNA. In some embodiments, the CRISPR effector protein is a Cas13a (C2c2) protein, a Cas13b protein, a Cas13c protein, or a Cas13d protein. In some embodiments, the CRISPR effector protein is a Cas9 protein. In some embodiments, the Cas9 protein is a S. aureus Cas9 (SauCas9) or a C. jejuni Cas9 (CjeCas9). In some embodiments, the Cas9 protein is a S. pyogenes Cas9 (SpyCas9) and the method comprises contacting the biological sample with a DNA oligonucleotide comprising the cognate PAM sequence (a PAMmer).

In some embodiments, the extended nucleic acid molecule comprises a cleavage site. In some embodiments, the extended concatemer comprises a cleavage site. In some embodiments, the cleavage site is a restriction digestion cleavage site recognized by a restriction enzyme. In some embodiments, the cleavage site is recognized by a DNAzyme.

In some embodiments, the method further comprises cleaving the extended concatemer using a DNA oligonucleotide. In some embodiments, the DNA oligonucleotide is a DNAzyme. In some embodiments, cleaving the extended concatemer comprises chemical cleavage. In some embodiments, the extended concatemer comprises a disulfide bond. In some embodiments, the chemical cleavage is performed using a reducing agent. In some embodiments, the reducing agent is DTT or THPP.

In some embodiments, the target RNA is a microRNA (miRNA). In some embodiments, the target RNA is a messenger (mRNA). In some embodiments, the target RNA is not a messenger (mRNA).

In some embodiments, the target recognition sequence at the 3′ end of the target RNA is unique to the target RNA. In some embodiments, the target recognition sequence is not a poly T sequence. In some embodiments, the target recognition sequence is not a poly A sequence.

In some embodiments, the polymerase is a strand-displacing polymerase selected from the group consisting of phi29 DNA polymerases, Bst DNA polymerases, and Bsu DNA polymerases.

In some embodiments, the method comprises imaging the extended nucleic acid molecule. In some embodiments, the method comprises imaging the extended concatemer. In some embodiments, the imaging comprises detecting a signal associated with a detectably labeled probe that directly or indirectly binds to the extended nucleic acid molecule and/or the extended concatemer. In some embodiments, the detectably labeled probe is a fluorescently labeled probe.

In some embodiments, the sequence of the extended nucleic acid molecule or the extended concatemer is analyzed by sequential hybridization, sequencing by hybridization, sequencing by ligation, sequencing-by-synthesis (SBS), sequencing-by-avidity (SBA), sequencing-by-binding (SBB), or a combination thereof. In some embodiments, the sequence of the extended nucleic acid molecule or extended concatemer is analyzed by single nucleotide sequencing by synthesis.

In some embodiments, the sequence of the extended nucleic acid molecule or the extended concatemer comprises one or more barcode sequences or complements thereof. In some embodiments, the one or more barcode sequences or complements thereof correspond to the target RNA. In some embodiments, each barcode sequence or complement thereof is assigned a series of signal codes that identifies the barcode sequence or complement thereof, and wherein detecting the barcode sequences or complements thereof comprises decoding the barcode sequences of complements thereof by detecting the corresponding sequences of signal codes detected from sequential hybridization, detection, and removal of sequential pools of intermediate probes and a universal pool of detectably labeled probes. In some embodiments, the series of signal codes are fluorophore sequences assigned to the corresponding barcode sequences or complements thereof. In some embodiments, the detectably labeled probes are fluorescently labeled. In some embodiments, a detectably labeled probe of the universal pool of detectably labeled probes binds to the extended concatemer at two different hybridization regions.

In some embodiments, the target RNA is in a biological sample. In some embodiments, the extended nucleic acid molecule or the extended concatemer is analyzed at a location in the biological sample or a matrix embedding the biological sample. In some embodiments, the target RNA is attached directly or indirectly to the biological sample or to a matrix embedding the biological sample. In some embodiments, the target RNA is crosslinked in the biological sample or in a matrix embedding the biological sample. In some embodiments, the extended nucleic acid molecule or the extended concatemer is immobilized in the biological sample and/or crosslinked to one or more other molecules in the biological sample. In some embodiments, the biological sample is a fixed and/or permeabilized biological sample. In some embodiments, the biological sample is a cell or tissue sample. In some embodiments, the biological sample is a formalin-fixed, paraffin-embedded (FFPE) tissue sample, a frozen tissue sample, or a fresh tissue sample. In some embodiments, the tissue sample is a tissue slice between about 1 μm and about 50 μm in thickness, optionally wherein the tissue slice is between about 5 μm and about 35 μm in thickness. In some embodiments, the biological sample is crosslinked. In some embodiments, the biological sample is embedded in a hydrogel matrix. In some embodiments, the biological sample is not embedded in a hydrogel matrix. In some embodiments, the biological sample is cleared.

In some embodiments, the biological sample comprises a non-nucleic acid target analyte and the biological sample is contacted with a labeling agent comprising a reporter oligonucleotide. In some instances, the method further comprises hybridizing an extension probe to a reporter oligonucleotide attached to the labeling agent, wherein the extension probe comprises (i) a target recognition sequence complementary to a target sequence of the reporter oligonucleotide, and (ii) a detection sequence; extending the reporter oligonucleotide with a polymerase using the extension probe as a template, thereby generating an extended nucleic acid molecule. In some embodiments, the method comprises performing further extension of the extended nucleic acid molecule using a plurality of additional extension probes, wherein an additional extension probe of the plurality of additional extension probe comprises (i) at least a portion of the detection sequence of the extension probe or a complement thereof, and (ii) an amplification sequence, thereby generating an extended concatemer comprising the reporter oligonucleotide, the detection sequence or a complement thereof and the amplification sequence or a complement thereof. In some embodiments, generating the a labeling agent attached to the extended concatemer is performed in situ. In some embodiments, the extended concatemer is generated in vitro. In some embodiments, extension of the reporter oligonucleotide is performed in a biological sample (e.g., a cell or tissue sample). In some embodiments, the extension and further extension is performed after the labeling agent binds to an analyte in a biological sample (e.g., a cell or tissue sample).

In other aspects, provided herein is a system for nucleic acid processing, comprising: (a) an extension probe, wherein the extension probe comprises i) a target recognition sequence complementary to a target sequence at the 3′ end of a target RNA, and ii) a detection sequence; (b) a plurality of additional extension probes, wherein an additional extension probe of the plurality of additional extension probes comprises: i) the detection sequence or a complement thereof, and ii) an amplification sequence; and (c) a polymerase for performing an extension reaction to generate an extended concatemer.

In other aspects, provided herein is a system for nucleic acid processing, comprising: (a) a complex comprising a guide nucleic acid and an RNA-cutting enzyme; (b) an extension probe, wherein the extension probe comprises (i) a target recognition sequence complementary to a target sequence at the 3′ end of a target RNA, and (ii) a detection sequence; (c) a plurality of additional extension probes, wherein an additional extension probe of the plurality of additional extension probes comprises: (i) the detection sequence or a complement thereof, and (ii) an amplification sequence; and (d) a polymerase for performing an extension reaction to generate an extended concatemer.

In some embodiments, the RNA-cutting enzyme is an Argonaute protein. In some embodiments, the Argonaute protein is an RNA-guided Argonautre, and the guide nucleic acid is an RNA molecule. In some embodiments, the Argonaute protein is a DNA-guided Argonautre, and the guide nucleic acid is a DNA molecule.

In some embodiments, the RNA-cutting enzyme is a CRISPR effector protein, and the guide nucleic acid is a CRISPR guide RNA comprising a spacer sequence, wherein the spacer sequence hybridizes to the target RNA.

In other aspects, provided herein is a system for nucleic acid processing, comprising: (a) a deoxyribonucleic acid (DNA) oligonucleotide, wherein the DNA oligonucleotide is complementary to an oligonucleotide hybridization region in a target ribonucleic acid (RNA); (b) a plurality of additional extension probes, wherein an additional extension probe of the plurality of additional extension probes comprises: (i) the detection sequence or a complement thereof, and (ii) an amplification sequence; (c) an extension probe, wherein the extension probe comprises (i) a target recognition sequence complementary to a target sequence at the 3′ end of the target RNA, and (ii) a detection sequence; and (d) a polymerase for performing an extension reaction of the cleaved target RNA.

In some embodiments, the system further comprises an RNase H for cleaving the oligonucleotide hybridization region of the target RNA.

In some embodiments, the DNA oligonucleotide is a DNAzyme.

In some embodiments, the polymerase is a strand-displacing polymerase selected from the group consisting of phi29 DNA polymerases, Bst DNA polymerases, and Bsu DNA polymerases.

In some embodiments, the extension probe comprises a stem-loop structure.

In some embodiments, the additional extension probe comprises a stem-loop structure.

In some embodiments, the extension probe comprises a stopper molecule or a stopper modification.

In some embodiments, the additional extension probe comprises a stopper molecule or a stopper modification.

In some embodiments, the extension probe is a linear oligonucleotide.

In some embodiments, the additional extension probe is a linear oligonucleotide.

In some embodiments, the system further comprises reagents for performing sequential hybridization, sequencing by hybridization, sequencing by ligation, sequencing-by-synthesis, sequencing-by-avidity, sequencing-by-binding, or a combination thereof. In some embodiments, the reagents for performing sequential hybridizing comprise a pool of detectably labeled probes.

In some embodiments, the system further comprises a plurality of free nucleotides.

In other aspects, provided herein is a kit for analyzing a biological sample, comprising: (a) a nucleic acid oligonucleotide, wherein the nucleic acid oligonucleotide is complementary to an oligonucleotide hybridization region in a target ribonucleic acid (RNA); (b) an extension probe, wherein the extension probe comprises (i) a target recognition sequence complementary to a target sequence at the 3′ end of the target RNA, and (ii) a detection sequence; (c) an RNase H for cleaving the oligonucleotide hybridization region of the target RNA when hybridized to the nucleic acid oligonucleotide; and (d) a polymerase for performing extension of the cleaved target RNA using the detection sequence of the extension probe as template.

In some embodiments, the kit further comprises a plurality of additional extension probes, wherein an additional extension probe of the plurality of additional extension probes comprises: (i) the detection sequence or a complement thereof, and (ii) an amplification sequence.

In some embodiments, the oligonucleotide hybridization region and the target sequence overlap by about 8 to about 12 nucleotides.

In some embodiments, the kit further comprises reagents for performing sequencing by ligation, sequencing by synthesis, sequencing by binding, sequencing by avidity or a combination thereof.

In some embodiments, the kit further comprises a universal pool of detectably labeled probes.

### BRIEF DESCRIPTION OF THE DRAWINGS

The drawings illustrate certain features and advantages of this disclosure. These embodiments are not intended to limit the scope of the appended claims in any manner.

FIG. 1 provides a schematic illustration of a method for target extension using an extension probe and an additional extension probe.

FIG. 2A provides a schematic illustration of hybridizing a nucleic acid oligonucleotide to a target RNA for RNase H cleavage of the target RNA, then hybridizing an extension probe with a stem loop structure to the cleaved target RNA to extend the cleaved target RNA. As shown in the figure, in some cases, the oligonucleotide hybridization region overlaps with the target sequence for the extension probe, such as an extension probe comprising a stem loop structure as depicted.

FIG. 2B provides a schematic illustration of further extension of the extended nucleic acid molecule from FIG. 2A using an additional extension probe. The additional extension probe comprises an amplification sequence that is the same as the detection sequence of the extension probe (FIG. 2B) or an amplification sequence that is different from the detection sequence of the extension probe. Further extension of extended nucleic acid molecule using two or more additional nucleic acid molecules as shown in FIG. 2C results in generation of an extended concatemer.

FIG. 3A provides a schematic illustration of hybridizing a nucleic acid oligonucleotide to a target RNA for RNase H cleavage of the target RNA, then hybridizing a linear extension probe to the cleaved target RNA to extend the cleaved target RNA. As shown in the figure, in some instances, the oligonucleotide hybridization region overlaps with the target sequence for the extension probe, such as a linear extension probe as depicted.

FIG. 3B provides a schematic illustration of a plurality of additional linear extension probes used for further extension of the extended nucleic acid molecule from FIG. 3A, wherein each additional extension probe comprises an adapter sequence at least one end.

FIG. 4A provides a schematic illustration of extending the reporter oligonucleotide and attaching (e.g., conjugating) it to a labeling agent for detecting a non-nucleic acid analyte in a sample.

FIG. 4B provides a schematic illustration of extending a reporter oligonucleotide attached (e.g., conjugated) to a labeling agent for detecting a non-nucleic acid analyte in a sample.

FIG. 4C provides a schematic illustration of hybridizing an extension probe to a reporter oligonucleotide of a labelling agent bound to an analyte in a sample and extending the reporter oligonucleotide.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Provided herein are methods, compositions, kits, and systems for performing an extension reaction of a target nucleic acid (e.g., target RNA). In some aspects, the target nucleic acid is extended to generate an amplification product. In some aspects, a cleaved target nucleic acid is extended to generate an amplification product. For example, a generated extension product is an extended nucleic acid molecule or an extended concatemer comprising the target RNA or a portion thereof and one or more additional sequences added by extension. In some embodiments, the one or more additional sequences added by extension comprises a detection sequence. In some embodiments, the one or more additional sequences added by extension comprises an amplification sequence. In some embodiments, the one or more additional sequences added by extension comprises two or more copies of a barcode sequence. In some aspects, the extension is performed using an extension probe and optionally, one or more additional extension probes. In some aspects, the extension adds a detection sequence to the target nucleic acid (e.g., a target RNA) or a portion thereof. In some aspects, the extension adds an amplification sequence to the target nucleic acid (e.g., a target RNA) or a portion thereof. In some aspects, the method comprises cleaving the target nucleic acid prior to extension. In some embodiments, cleaving of the target nucleic acid allows extension to be performed using an extension probe that is designed to hybridize to a particular sequence of the target RNA. Without cleavage, in some cases, the position of the free 3′ of the target RNA is not aligned with the extension probe to be used as a template for the extension reaction.

In some embodiments, the cleaving of the target nucleic acid is performed enzymatically. In some embodiments, the cleaving of the target nucleic acid is performed by using an RNase H. For example, the method comprises targeting of RNase H activity to a particular region in a target RNA that is adjacent to or overlapping with a target sequence for the extension probe. For example, a nucleic acid oligonucleotide is designed to hybridize to a complementary oligonucleotide hybridization region in the target RNA. In some embodiments, the nucleic acid oligonucleotide is a DNA oligonucleotide, or comprises at least 3, 4, 5, 6, 10, or more contiguous DNA bases to provide a DNA-RNA duplex upon hybridization to the target RNA. Formation of the DNA-RNA duplex allows RNase H to cleave the target RNA within the duplex region (e.g., within the oligonucleotide hybridization region) to generate a cleaved target RNA. Optionally, one or more washes are performed to remove the nucleic acid oligonucleotide and/or the RNase H before contacting the biological sample with the extension probe. In some embodiments, the cleaving of the target nucleic acid is performed by using an RNA-cutting enzyme and a guide nucleic acid. In some embodiments, the extension probe hybridizes to the cleaved target RNA, and the cleaved target RNA is extended using at least a portion of the extension probe as template.

Provided herein is a method comprising contacting the biological sample with a nucleic acid oligonucleotide, wherein the nucleic acid oligonucleotide hybridizes to an oligonucleotide hybridization region in a target ribonucleic acid (RNA); contacting the biological sample with an RNase H to cleave the target RNA in the oligonucleotide hybridization region to generate a cleaved target RNA; and after cleavage, hybridizing an extension probe to a target sequence in the cleaved target RNA.

In some aspects, the methods for extension of target RNAs to generate a plurality of amplification products provided herein simplify the library preparation process. In some aspects, the methods for extension of a target RNA to generate an amplification product provided herein eliminates the need for a ligation. In some cases, compared to amplification reactions that require a circular or circularizable probe to serve as a circular template for rolling circle amplification, the provided methods removes the need for a relatively long nucleic acid probe molecule that serves as the circular template. In contrast, extension of the target RNA as provided herein is in some aspects is simpler, requires fewer reagents, and/or requires shorter length oligonucleotide (e.g., extension probes) to be manufactured. In some aspects, the extension of the target RNA as provided herein is in some aspects is well suited for automation (e.g., on an instrument) and/or reduces hands-on time from a user. In some cases, the extension of the target RNA is an efficient reaction, for example, more efficient compared to an RCA reaction. In some cases, the extension of the target RNA achieves high sensitivity for analyte detection. For example, without the requirement for performing a ligation in the provided methods for target RNA extension, the simplified workflow achieves improved efficiency and/or sensitivity compared to a method for amplification that requires one or more ligations. In some aspects, the extension of the target RNA can be tuned to achieve a desired level of amplification (e.g., by controlling the concentration of extension probes and additional extension probes provided for the extension reaction).

Another advantage of the provided methods in some aspects is that the resulting amplification product (e.g., the extended nucleic acid molecule or the extended concatemer) is covalently attached to their respective target RNAs (or a portion thereof for extending cleaved target RNAs). In some aspects, this increases positional stability of the molecule in the biological sample and improves accuracy of localization for detected signals based on detection of the generated extension product, e.g., an extended nucleic acid molecule or an extended concatemer comprising the target RNA or a portion thereof. In some cases, by cutting the site next to the target recognition sequence where the extension probe binds the target RNA, there may be a reduction of the tension and/or hinderance from a heavily entangled mRNA in its micro-environment, and may promote a more relaxed and uniform milieu for the polymerase to perform extension. In some aspects, an advantage of the provided methods is that the sequences for detection (e.g., detection sequence or amplification sequence or complements thereof) in the extension product (e.g., the extended nucleic acid molecule or the extended concatemer) is covalently attached to their respective target RNAs (or a portion thereof). In some aspects, the sequences for detection are in the same molecule as the target RNA as it is extended to include the additional sequences for detection. In some aspects, directly adding the sequences for detection and covalently attaching them to the original target RNA allows for detection by imaging the molecules in a preserved spatial location. In some cases, if detection sequences are not covalently attached to the target RNA (e.g., are not part of the same continuous molecule), the position and spatial location of the target RNA may not be preserved. In some aspects, the extension product (e.g., the extended nucleic acid molecule or the extended concatemer) is covalently attached to their respective target RNAs (or a portion thereof) during detection. In some aspects, the extension product (e.g., the extended nucleic acid molecule or the extended concatemer) is not cleaved from the respective target RNAs (or a portion thereof) during detection. In some aspects, the extension product (e.g., the extended nucleic acid molecule or the extended concatemer) is a single continuous nucleic acid molecule comprising the target RNA (or a portion thereof) during detection.

In some aspects, the present application provides various designs for extension probes and additional extension probes to provide a template sequence in the extension of the target nucleic acid or portion thereof.

Additional aspects of the methods, compositions, kits, and systems disclosed herein are described in the sections below.

### II. Methods for Target Extension

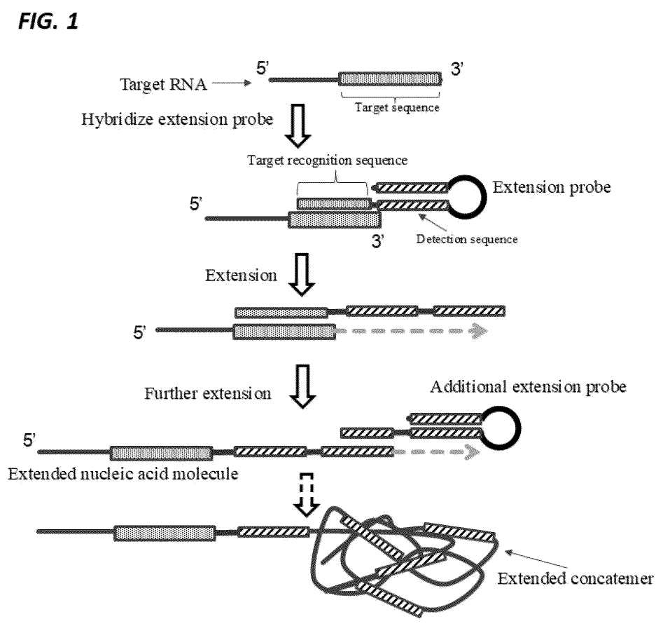

In some aspects, provided herein are methods for target RNA extension. In some instances, the extension is performed using an extension probe as template. In some cases, the generated extended nucleic acid molecule comprises a detection sequence or a complement thereof. In some aspects, the generated extended nucleic acid molecule is further extended by a polymerase to add one or more additional amplification sequence(s) or complements thereof. In some cases, the generated extended concatemer comprises a detection sequence or a complement thereof and one or more additional amplification sequence(s) or complements thereof that are covalently attached to their respective target RNAs, as illustrated in FIG. 1. In FIG. 1, the extended probe and additional extended probe may have a stem loop structure as illustrated or may be linear oligonucleotides. In some aspects, the generated extended nucleic acid molecule comprises a complement of the detection sequence from the extension probe used as a template. In some aspects, the generated extended nucleic acid molecule comprises at least two copies of the detection sequence (or a complement thereof) from the extension probe used as a template. In some aspects, the detection sequence is a barcode sequence corresponding to the target RNA, or a complement thereof. In some aspects, the generated extended nucleic acid molecule is further extended using a plurality of additional extension probes. In some aspects, an additional extension probe comprises at least a portion of the detection sequence of the extension probe or a complement thereof, and an amplification sequence. An additional extension probe serves as template to further extend the extended nucleic acid molecule to generate an extended concatemer which comprises the target RNA, the detection sequence or a complement thereof and the amplification sequence or a complement thereof. In some instances, the generated extended concatemer comprises at least two copies of the amplification sequence or complements thereof.

Provided herein is a method of nucleic acid processing comprising hybridizing an extension probe to a target RNA, wherein the extension probe comprises i) a target recognition sequence complementary to a target sequence at the 3′ end of the target RNA, and ii) a detection sequence; extending the target RNA with a polymerase using the extension probe as a template, thereby generating an extended nucleic acid molecule; and performing further extension of the extended nucleic acid molecule using a plurality of additional extension probes, wherein an additional extension probe of the plurality of additional extension probe comprises i) at least a portion of the detection sequence of the extension probe or a complement thereof, and ii) an amplification sequence; thereby generating an extended concatemer comprising the target RNA or a portion thereof, the detection sequence or a complement thereof and the amplification sequence or a complement thereof.

Provided herein is a method of nucleic acid processing comprising generating a cleaved target RNA; hybridizing an extension probe to the cleaved target RNA, wherein the extension probe comprises i) a target recognition sequence complementary to a target sequence in the target RNA, and ii) a detection sequence; and extending the cleaved target RNA with a polymerase using the extension probe as a template, thereby generating an extended nucleic acid molecule. In some embodiments, generating the cleaved target RNA comprises hybridizing a deoxyribonucleic acid (DNA) oligonucleotide to an oligonucleotide hybridization region in a target ribonucleic acid (RNA) and cleaving the target RNA in the oligonucleotide hybridization region, thereby generating a cleaved target RNA. In some embodiments, generating the cleaved target RNA comprises contacting the target RNA with a complex comprising a guide nucleic acid and an RNA-cutting enzyme to guide cutting of a guide target sequence in the target RNA by the RNA-cutting enzyme. In some instances, the RNA-cutting enzyme is an Argonaute protein. In some instances, the RNA-cutting enzyme is a CRISPR effector protein.

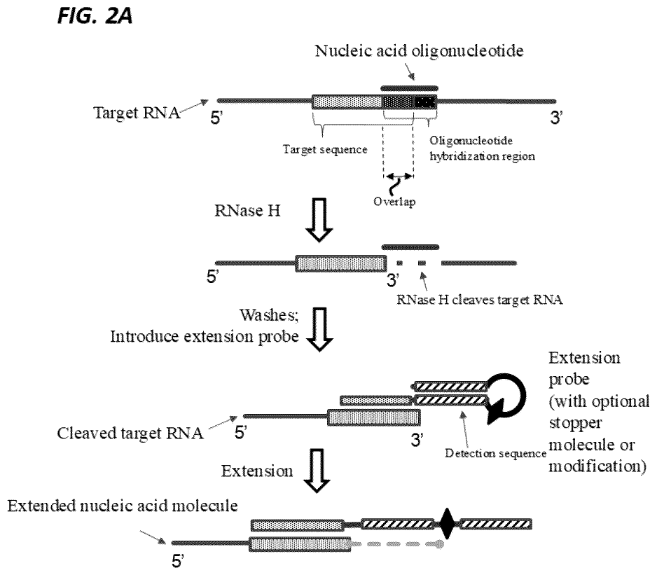

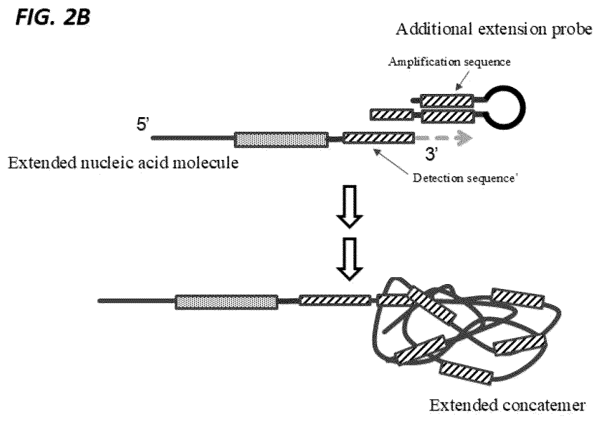

In some cases, the target nucleic acid is an RNA that is cleaved prior to extension. In some cases, the target RNA is cleaved enzymatically, e.g., using an RNase H. In some cases, the target RNA is cleaved using a DNA oligonucleotide, e.g., using a DNAzyme. In some cases, a nucleic acid oligonucleotide hybridizes to the target RNA to provide a DNA-RNA duplex upon hybridization to the target RNA, for cleavage of the target RNA by the RNase H. As illustrated in FIG. 2A, in some embodiments, the method comprises hybridizing a nucleic acid oligonucleotide to an oligonucleotide hybridization region in a target RNA to form a DNA-RNA duplex with at least a portion of the oligonucleotide hybridization region. In some instances, RNase H cleaves the target RNA within the oligonucleotide hybridization region, as shown in FIG. 2A. While it is depicted that the cleavage of the target RNA is performed using RNase H and the nucleic acid oligonucleotide in FIG. 2A, other reagents such as a DNAzyme or a complex comprising a guide nucleic acid and an RNA-cutting enzyme can be similarly used. In some embodiments, one or more washes are then performed, and the cleaved target RNA is contacted with an extension probe. Although an extension probe comprising a stem loop structure (e.g., a hairpin) is illustrated in FIG. 1, the extension probe can be a linear nucleic acid molecule that can be used to provide a template for the extension by the polymerase. After incubating the sample to allow the extension probe to hybridize to the target RNA, extension of a 3′ end of the target RNA is performed using a sequence of the extension probe as template. FIG. 2A also illustrates how the target sequence for the extension probe can overlap with the oligonucleotide hybridization region (e.g., by 1, 2, 3, 4, 5, 6, 7, 8, 9, 10, or more nucleotides).

In some embodiments, provided herein is a method of analyzing a biological sample, comprising: a) hybridizing a deoxyribonucleic acid (DNA) oligonucleotide to an oligonucleotide hybridization region in a target ribonucleic acid (RNA); b) cleaving the target RNA in the oligonucleotide hybridization region, thereby generating a cleaved target RNA; c) hybridizing an extension probe to the cleaved target RNA, wherein the extension probe comprises i) a target recognition sequence complementary to a target sequence in the target RNA, and ii) a detection sequence; and d) extending the cleaved target RNA with a polymerase using the extension probe as a template, thereby generating an extended nucleic acid molecule. In some embodiments, the extended nucleic acid molecule is contacted with a plurality of additional extension probes, wherein an additional extension probe of the plurality of additional extension probe comprises i) at least a portion of the detection sequence of the extension probe or a complement thereof, and ii) an amplification sequence; and the extended nucleic acid molecule is extended using an additional extension probe of the plurality of additional extension probes as template, thereby generated an extended concatemer.

In some embodiments, the method comprises performing a wash after hybridizing the extension probe to the target sequence and/or hybridizing an additional extension probe to a sequence in the extended nucleic acid molecule. In some embodiments, the method comprises performing a wash after the extension probe hybridizes to the cleaved target RNA. In some embodiments, the sequence of the extension probe at the 5′ end comprises a sequence complementary to a target sequence at the 3′ end of the target RNA.

In some embodiments, provided herein is a method of analyzing a biological sample, comprising: hybridizing an extension probe to a target RNA, wherein the extension probe comprises i) a target recognition sequence complementary to a target sequence at the 3′ end of the target RNA, and ii) a detection sequence, b) extending the target RNA with a polymerase using the extension probe as a template, thereby generating an extended nucleic acid molecule; and c) performing further extension of the extended nucleic acid molecule using a plurality of additional extension probes, wherein an additional extension probe of the plurality of additional extension probe comprises i) at least a portion of the detection sequence of the extension probe or a complement thereof, and ii) an amplification sequence; thereby generating an extended concatemer comprising the target RNA or a portion thereof, the detection sequence or a complement thereof and the amplification sequence or a complement thereof.

### A. Extension Probes

Disclosed herein in some aspects are extension probes that hybridize to another nucleic acid molecule (e.g., the target RNA) to provide a template for an extension reaction. In some embodiments, the extension probes are introduced into a cell comprising the target RNA or used to otherwise contact a biological sample such as a tissue sample. In some aspects, the extension probe comprises a target recognition sequence complementary to a target sequence in the target RNA or a cleaved portion thereof. In some aspects, the extension probe comprises a detection sequence corresponding to the target RNA. Disclosed herein in some aspects are additional extension probes that hybridize to an extended nucleic acid molecule comprising a target RNA or a portion thereof. In some embodiments, the additional extension probes are introduced into a cell or used to otherwise contact a biological sample such as a tissue sample. In some aspects, the additional extension probe comprises at least a portion of the detection sequence of the extension probe used to generate the extended nucleic acid molecule from extending the target RNA by a polymerase. In some aspects, the additional extension probe comprises at least a portion of a complement of the detection sequence of the extension probe. In some aspects, extension probes and additional extension probes provide a template for a polymerase to perform an extension of the target RNA or a nucleic acid molecule comprising the target RNA or a cleaved portion thereof.

The probes (e.g., extension probes, additional extension probes) may comprise any of a variety of entities that can hybridize to a nucleic acid, typically by Watson-Crick base pairing, such as DNA, RNA, LNA, PNA, etc. In some aspects, the extension probe comprises a sequence (e.g., hybridization region such as a target recognition sequence) that directly or indirectly binds to at least a portion of the target nucleic acid (e.g., a target RNA). In some instances, the extension probe binds to a specific target nucleic acid (e.g., an mRNA, or other nucleic acids as discussed herein). In some embodiments, extended nucleic acid molecules or extended concatemers generated from using the extension probes are detected using a detectable label, by using detectably labelled nucleic acid probes that directly or indirectly bind to the extended nucleic acid molecules or extended concatemers or sequences thereof.

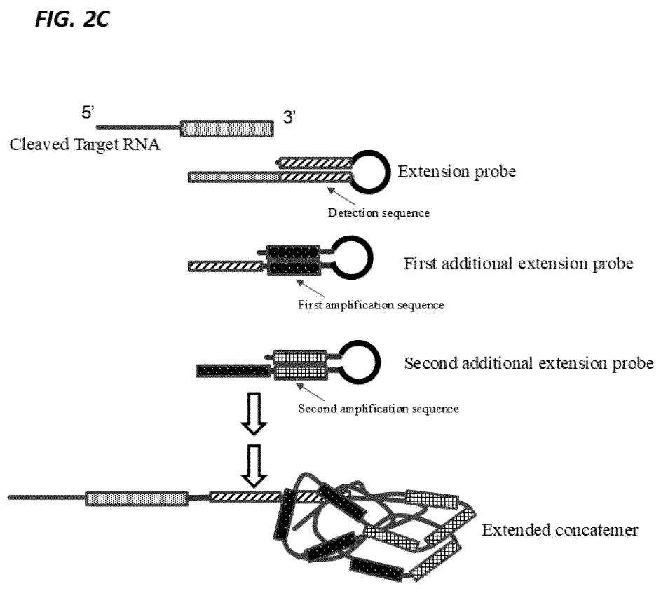

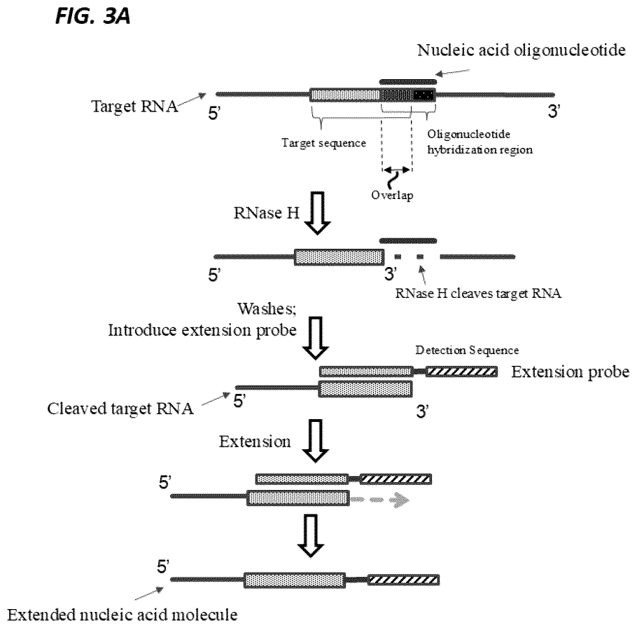

In some embodiments, more than one type of extension probe is contacted with a sample. In some instances, the extension probe is hybridized to the target RNA or a portion thereof and serves as a template for extension of the target RNA or a portion thereof. In some instances, the additional extension probe is hybridized to an extended target RNA (e.g., extended nucleic acid molecule comprising the target RNA or a portion thereof) and serves as a template for further extension. In some embodiments, the extension probe comprises a stem loop structure. In some embodiments, the extension probe does not comprise a stem loop structure. In some embodiments, the extension by the polymerase is performed without the use of a catalytic hairpin. In some embodiments, the extension probe is a linear nucleic acid molecule (as shown in FIG. 3A). In some embodiments, the additional extension probe comprises a stem loop structure. In some embodiments, the additional extension probe does not comprise a loop structure. In some embodiments, the additional extension probe is a linear nucleic acid molecule.

In some embodiments, the extension probe comprises a detection sequence. In some embodiments, the detection sequence is in the stem of an extension probe with a stem loop structure. In some embodiments, the detection sequence is in the loop of an extension probe with a stem loop structure. In some aspects, the detection sequence is a barcode sequence that identifies the target RNA. In some aspects, the detection sequence comprises a barcode sequence. In some embodiments, the barcode sequence comprises two or more sub-barcodes that together function as a single barcode. In some examples, a detection sequence comprises two or more sub-barcodes that are separated by one or more non-barcode sequences. In some examples, a detection sequence comprises two or more sub-barcodes that are overlapping.

In some embodiments, the additional extension probe comprises an amplification sequence. In some embodiments, the amplification sequence is in the stem of an additional extension probe with a stem loop structure. In some embodiments, the amplification sequence is in the loop of an additional extension probe with a stem loop structure. In some aspects, the amplification sequence is a barcode sequence that identifies the target RNA. In some aspects, the amplification sequence comprises a barcode sequence. In some embodiments, the barcode sequence comprises two or more sub-barcodes that together function as a single barcode. In some examples, an amplification sequence comprises two or more sub-barcodes that are separated by one or more non-barcode sequences. In some examples, an amplification sequence comprises two or more sub-barcodes that are overlapping (e.g., partially overlapping at the end). In some instances, the additional extension probe comprises two or more copies of the detection sequence of the extension probe or a complement thereof. In some instances, the amplification sequence of the additional extension probe comprises the same sequence as the detection sequence. In some instances, the additional extension probe comprises the same sequence as the detection sequence. In some instances, the additional extension probe comprises a complement of the detection sequence.

In some aspects, the extended concatemer comprises a plurality of amplification sequences or complements thereof that collectively identifies the target RNA. In some embodiments, the amplification sequence of an additional extension probe comprises the same sequence as the detection sequence of the extension probe used to extend the target RNA or a portion thereof. In some instances, the amplification sequence is different from the detection sequence. In some instances, the additional extension probe comprises two or more copies of the detection sequence of the extension probe or a complement thereof. In some embodiments, a target RNA is extended using a plurality of additional extension probes, wherein the plurality of additional extension probes comprises a first additional extension probe and a second additional extension probe, wherein the first additional extension probe and the second additional extension probe comprises different amplification sequences. In some embodiments, an additional extension probe comprises an amplification sequence that is the same as the detection sequence of the extension probe (FIG. 2B) or an amplification sequence that is different from the detection sequence of the extension probe. Further extension of the extended nucleic acid molecule using two or more additional nucleic acid molecules as shown in FIG. 2C results in generation of an extended concatemer. In some instances, the additional extension probe comprises at least two copies of an amplification sequence. In some aspects, the design of the additional extension probe including the number of copies of amplification sequence is tuned for a desired copy number of the amplification to be detected in the generated concatemer.

In some embodiments, at least 2, at least 5, at least 10, at least 25, at least 50, at least 75, at least 100, at least 300, at least 1,000, at least 3,000, at least 10,000, at least 30,000, at least 50,000, at least 100,000, at least 250,000, at least 500,000, or at least 1,000,000 distinguishable extension probes and/or additional extension probes are contacted with a sample, e.g., simultaneously or sequentially in any suitable order. In some embodiments, at least 2, at least 5, at least 10, at least 25, at least 50, at least 75, at least 100, at least 300, at least 1,000, at least 3,000, at least 10,000, at least 30,000, at least 50,000, at least 100,000, at least 250,000, at least 500,000, or at least 1,000,000 distinguishable additional extension probes and/or additional extension probes are contacted with a sample, e.g., simultaneously or sequentially in any suitable order. In some embodiments, at least 500, at least 1,000, at least 2,000, at least 3,000 distinguishable extension probes and/or additional extension probes are contacted with a sample. In some embodiments, a plurality of extension probes comprises at least two different extension probes corresponding to two different target RNAs. In some embodiments, at least 500, at least 1,000, at least 2,000, at least 3,000 distinguishable additional extension probes are contacted with a sample. In some embodiments, a plurality of distinguishable extension probes are complementary to the same target RNA. For example, at least 2, 3, 4, 5, 6, 7, 8, 9, or 10 distinguishable extension probes each hybridize to the same target sequence of a target RNA. In some aspects, the different extension probes may be used to introduce two or more different detection sequences to two or more different molecules of the same target RNA. In some cases, the different detection sequences or complements thereof are detected at different times such that optical crowding is reduced.

The target recognition sequence of the extension probe may be of any length, and multiple recognition sequences in the same or different extension probes may be of the same or different lengths. For instance, the target recognition sequence may be at least 20, at least 25, at least 30, at least 35, at least 40, or at least 50 nucleotides in length. In some embodiments, the target recognition sequence may be no more than 48, no more than 45, or no more than 40 nucleotides in length. Combinations of any of these are also possible, e.g., the recognition sequence may have a length of between 25 and 40, between 30 and 45, or between 20 and 48 nucleotides, etc. In some embodiments, the target recognition sequence is at least 95%, at least 98%, at least 99%, or at least 100% complementary to the target sequence in the target RNA.

In some embodiments, the target recognition sequence of an extension probe is positioned 3′ to any detection sequence (e.g., barcode sequence) in the extension probe. In some embodiments, the target recognition sequence comprises a sequence that is substantially complementary to a portion of a target nucleic acid (e.g., a target sequence). In some embodiments, the target recognition sequence and the target sequence are at least 50%, at least 60%, at least 70%, at least 75%, at least 80%, at least 85%, at least 90%, at least 92%, at least 94%, at least 95%, at least 96%, at least 97%, at least 98%, at least 99%, or 100% complementary.

In some aspects, the extension probe comprises a stem loop structure. For example, the extension probe comprises a stem sequence, a loop sequence, and a complement of the stem sequence to form the stem loop structure. In some cases, the extension probe comprises one or more additional sequences outside of the stem and loop sequences. In some embodiments, the extension probe comprises a loop domain between the detection sequence and a complement of the detection sequence. In some embodiments, the extension probe comprises a loop domain comprising the detection sequence. In some embodiments, the extension probe comprises from 5′ to 3′: the complement of the detection sequence-the loop domain-the detection sequence-the target recognition sequence. In some embodiments, the extension probe is a linear oligonucleotide comprising from 5′ to 3′: the detection sequence-the target recognition sequence.

In some embodiments, the 3′ end of the extension probe comprises a 3′ inverted dT to prevent extension by the polymerase. In some embodiments, the 3′ end of the additional extension probe comprises a 3′ inverted dT to prevent extension by the polymerase. In some embodiments, the 3′ end of the extension probe comprises a poly-T sequence (e.g., TTTTTTT) in a region that is not hybridized to the target RNA to prevent extension by the polymerase. In some embodiments, the 3′ end of the additional extension probe comprises a poly-T sequence in a region that is not hybridized to the target RNA to prevent extension by the polymerase (e.g., TTTTTTT). In some embodiments, the 3′ end of the extension probe comprises an overhang region that is not configured to hybridize to the target RNA. In some embodiments, the 3′ end of the additional extension probe comprises an overhang region that is not configured to hybridize to the target RNA.

In some aspects, the additional extension probe comprises a stem loop structure. For example, the additional extension probe comprises a stem sequence, a loop sequence, and a complement of the stem sequence to form the stem loop structure. In some cases, the additional extension probe comprises one or more additional sequences outside of the stem and loop sequences. In some embodiments, the additional extension probe comprises a loop domain between the amplification sequence and a complement of the amplification sequence. In some embodiments, the additional extension probe comprises from 5′ to 3′: a complement of the amplification sequence-the loop domain-the amplification sequence-the detection sequence or a complement thereof. In some embodiments, the additional extension probe is a linear oligonucleotide comprising from 5′ to 3′: the amplification sequence-at least a portion of the detection sequence or a complement thereof.

In some embodiments, the extension probe and/or the additional extension probe(s) are linear oligonucleotides. In some instances, the extension probe comprises a target recognition sequence at one end and an adapter sequence at the other end. In some cases, the adapter sequences or complements thereof generated by a polymerase serve as a sequence for binding of additional extension probes. In some aspects, “binding” as used herein refers to the coupling between two or more nucleic acids, e.g., oligonucleotides and/or polynucleotides. In some embodiments, the binding is indirect binding. In some embodiments, the binding is direct (e.g., binding comprising direct hybridization of nucleic acid sequences). The nature of the binding may vary. In some instances, a first nucleic acid sequence directly binds to a second nucleic acid sequence via hybridization of complementary sequences. In some instances, a first nucleic acid sequence indirectly binds to a second nucleic acid sequence via one or more intermediate nucleic acids. For example, an intermediate nucleic acid comprises a first region that binds to the first nucleic acid sequence and has a second region for binding to the second nucleic acid sequence, thereby forming a complex comprising the first and second nucleic acid sequences.

In some embodiments, the additional extension probe comprises an adapter sequence at both ends. In some instances, the adapter sequence at the ends of the additional extension probe are the same. In some instances, the adapter sequence at the ends of the additional extension probe are different (as shown in FIG. 3B). In some instances, the extension probe comprises from 3′ to 5′: the detection sequence-an amplification sequence-a first adapter sequence. In some instances, the additional extension probe comprises from 3′ to 5′: the first adapter sequence-an additional amplification sequence-a second adapter sequence. In some instances, the amplification sequence and the additional amplification sequence are different. In some cases, the amplification sequence and the additional amplification sequence are the same. In some instances, the first adapter sequence and the second adapter sequence are different. In some instances, the first adapter sequence and the second adapter sequence are the same.

In some aspects, the extension probe comprises at least two different detection sequences. In some aspects, the additional extension probe comprises at least two different amplification sequences. In some embodiments, an extension probe or an additional extension probe comprises 1, 2, 3, 4, 5, 6, 7, 8, 9, 10, 11, 12, 13, 14, 15 or more, 20 or more, 32 or more, 40 or more, or 50 or more barcode sequences. The barcode sequences may be positioned anywhere within the probe. If more than one barcode sequences are present, the barcode sequences may be positioned next to each other, and/or interspersed with other sequences. In some embodiments, two or more of the barcode sequences may also at least partially overlap. In some embodiments, two or more of the barcode sequences in the same probe do not overlap. In some embodiments, all of the barcode sequences in the same probe are separated from one another by at least a phosphodiester bond (e.g., they may be immediately adjacent to each other but do not overlap), such as 1, 2, 3, 4, 5, 6, 7, 8, 9, 10 or more nucleotides apart.

In some embodiments, the amplification sequence of the additional extension probe has the same sequence as the detection sequence of the extension probe. In some instances, the additional extension probe has the same sequence as the extension probe. In some instances, the extension probe is used as the additional extension probe for additional rounds of extension to generate the extended concatemer. In some cases, the extension probe and the additional extension probe comprises from 3′ to 5′: the target recognition sequence-the detection sequence-a complement of the detection sequence-at least a portion of a complement of the target recognition sequence or a portion thereof.

The detection sequence of the extension probe may be of any length, and multiple detection sequences in the same or different extension probes may be of the same or different lengths. In some instances, the detection sequence is at least 5, at least 10, at least 15, at least 20, at least 25, at least 30, at least 35, at least 40, at least 50 nucleotides in length. In some embodiments, the detection sequence is no more than 50, no more than 40, no more than 30, no more than 20, or no more than 15 nucleotides in length. Combinations of any of these are also possible, e.g., the detection sequence, in some instances, has a length of between 12 and 50, between 12 and 30, or between 15 and 20 nucleotides, etc.

The amplification sequence of the additional extension probe may be of any length, and multiple amplification sequences in the same or different additional extension probes may be of the same or different lengths. In some instances, the amplification sequence is at least 5, at least 10, at least 15, at least 20, at least 25, at least 30, at least 35, at least 40, at least 50 nucleotides in length in length. In some embodiments, the amplification sequence is no more than 50, no more than 40, no more than 30, no more than 20, or no more than 15 nucleotides in length. Combinations of any of these are also possible, e.g., the amplification sequence, in some instances, has a length of between 12 and 50, between 12 and 30, or between 15 and 20 nucleotides, etc.

In some aspects, the detection sequence and/or the amplification sequence is a barcode sequence. The barcode sequences, if present, may be of any length. If more than one barcode sequence is used, the barcode sequences may independently have the same or different lengths, such as at least 5, at least 10, at least 15, at least 20, at least 25, at least 30, at least 35, at least 40, at least 50 nucleotides in length. In some embodiments, the barcode sequence may be no more than 120, no more than 112, no more than 104, no more than 96, no more than 88, no more than 80, no more than 72, no more than 64, no more than 56, no more than 48, no more than 40, no more than 32, no more than 24, no more than 16, or no more than 8 nucleotides in length. Combinations of any of these are also possible, e.g., the barcode sequence may be between 5 and 10 nucleotides, between 8 and 15 nucleotides, etc.

The barcode sequence may be arbitrary or random. In certain cases, the barcode sequences are chosen so as to reduce or minimize homology with other components in a sample, e.g., such that the barcode sequences do not themselves bind to or hybridize with other nucleic acids suspected of being within the cell or other sample. In some embodiments, between a particular barcode sequence and another sequence (e.g., a cellular nucleic acid sequence in a sample or other barcode sequences in probes added to the sample), the homology may be less than 10%, less than 8%, less than 7%, less than 6%, less than 5%, less than 4%, less than 3%, less than 2%, or less than 1%. In some embodiments, the homology may be less than 20, 19, 18, 17, 16, 15, 14, 13, 12, 11, 10, 9, 8, 7, 6, 5, 4, 3, or 2 bases, and in some embodiments, the bases are consecutive bases.

The target recognition sequence of an extension probe is designed with reference to a target nucleic acid (e.g., a cellular RNA such as an mRNA) that is present or suspected of being present in a sample. In some embodiments, more than one target recognition sequence is used to identify a particular target RNA. In some embodiments, a first extension probe has a first target recognition sequence that is used to hybridize to a particular target RNA and a second extension probe has a second target recognition sequence that is used to hybridize to another target RNA. In some embodiments, multiple probes can be used, sequentially and/or simultaneously, that can bind to (e.g., hybridize to) different regions of different molecules of the same target RNA.

In some embodiments, the extension probe is a single stranded linear nucleic acid molecule. In some embodiments, the extension probe comprises a target recognition sequence and a sequence that does not hybridize to a target nucleic acid, such as a 5′ overhang, a 3′ overhang, and/or a linker or spacer (which may comprise a nucleic acid sequence or a non-nucleic acid moiety). In some embodiments, the sequence (e.g., the 5′ overhang, 3′ overhang, and/or linker or spacer) that is non-hybridizing to the target nucleic acid comprises the detection sequence. In some embodiments, the sequence (e.g., the 5′ overhang, 3′ overhang, and/or linker or spacer) that is non-hybridizing to the target nucleic acid is used as a template for extending the target nucleic acid (e.g., RNA).

In some embodiments, the additional extension probe is a single stranded linear nucleic acid molecule. In some embodiments, the additional extension probe comprises the detection sequence of a corresponding extension probe or a complement thereof and a sequence that does not hybridize to a target nucleic acid, such as a 5′ overhang, a 3′ overhang, and/or a linker or spacer (which may comprise a nucleic acid sequence or a non-nucleic acid moiety). In some embodiments, the sequence (e.g., the 5′ overhang, 3′ overhang, and/or linker or spacer) that is non-hybridizing to the extended nucleic acid molecule generated using a polymerase and the extension probe as template comprises the amplification sequence. In some embodiments, the sequence (e.g., the 5′ overhang, 3′ overhang, and/or linker or spacer) that is non-hybridizing to the target nucleic acid is used as a template for further extending the extended nucleic acid molecule.

In some embodiments, the extension probe and/or the additional extension is not ligated to other nucleic acid molecules. In some embodiments, the extension probe and/or the additional extension is not configured to be ligated to other nucleic acid molecules.

In some embodiments, the extension probe comprises a stopper molecule or a stopper modification. In some embodiments, the additional extension probe comprises a stopper molecule or a stopper modification. In some embodiments, extension of the extended nucleic acid molecule by the polymerase is terminated by the presence of a molecule or modification in the extension probe or additional extension probe that terminates polymerization. For example, the stopper molecule or the stopper modification prevents polymerization and halts extension of the extended nucleic acid molecule. In some cases, the stopper molecule or the stopper modification is located in the loop structure of the extension probe. In some cases the stopper molecule or the stopper modification is located in the loop structure of the additional extension probe. In some cases, the stopper molecule or the stopper modification is located at the end of a stem structure of the extension probe. In some cases, the stopper molecule or the stopper modification is located at the end of a stem structure of the additional extension probe.

In some instances, the stopper molecule or stopper modification comprises a chemical modification. For example, the chemical modification is a phosphoramidite. In some instances, the extension probe comprises a C3 spacer phosphoramidite. In some instances, the additional extension probe comprises a C3 spacer phosphoramidite. In some cases, the chemical modification is a sulfhydryl reactive group. In some instances, the chemical modification is an azide modification configured to be covalently linked to an alkyne through click chemistry. In some embodiments, the chemical modification is an amine or a carboxyl group. In some embodiments, the chemical modification comprises an azide. In some embodiments, the chemical modification is a dibenzocyclooctyne (DBCO) group or a bicyclononyne (BCN) group. In some examples, the stopper molecule or stopper modification is selected from a triethylene glycol (TEG), 18-atom hexa-ethylene glycol, adenylation, digoxigenin, cholesteryl-TEG, and 3-cyanovinylcarbazole (CNVK). In some instances, the extension probe and/or the additional extension probe comprises a synthetic non-DNA linker configured to terminate polymerization. In some cases, the extension probe and/or the additional extension probe comprises a non-natural nucleotide (e.g., iso-dG or iso-dC).

In some embodiments, polymerization is halted at a nucleotide in the extension probe where a complementary nucleotide for base pairing is not provided. In some instances, the stopper molecule is a first nucleotide and the extension and/or further extension is performed with a plurality of free nucleotides that lack a free nucleotide that base-pairs with the first nucleotide. For example, the stopper molecule is guanine, and the extension is performed with a plurality of free nucleotides that lacks cytosine. In some instances, the stopper molecule is cytosine, and the extension is performed with a plurality of free nucleotides that lacks guanine. In some instances, the stopper molecule is thymine, and the extension is performed with a plurality of free nucleotides that lacks adenine. In some instances, the stopper molecule is adenine, and the extension is performed with a plurality of free nucleotides that lacks thymine. In some instances, the extension probe comprises a stem loop structure, wherein a loop sequence of the loop of the structure is a plurality of the first nucleotide and a stem sequence of the stem loop structure is made of a plurality of nucleotides that is not the first nucleotide. For example, in an extension probe or additional extension probe, the loop comprises a first nucleotide and the stem is made of the other 3 nucleotides and in an extension reaction performed using a plurality of free nucleotides that lacks a nucleotide that base pairs with the first nucleotide, polymerization is halted.

In some embodiments, polymerization is halted at a nucleotide in the extension probe where a complementary nucleotide for base pairing comprises a terminating group. In some instances, the stopper molecule is a first nucleotide and the extension and/or further extension is performed with a plurality of free nucleotides comprising a terminating nucleotide that base-pairs with the first nucleotide. For example, the stopper molecule is guanine, and the extension is performed with a plurality of free nucleotides comprising a terminating cytosine. In some instances, the stopper molecule is cytosine, and the extension is performed with a plurality of free nucleotides comprising a terminating guanine. In some instances, the stopper molecule is thymine, and the extension is performed with a plurality of free nucleotides comprising a terminating adenine. In some instances, the stopper molecule is adenine, and the extension is performed with a plurality of free nucleotides comprising a terminating thymine. In some instances, the extension probe comprises a stem loop structure, wherein a loop sequence of the loop of the structure comprises a sequence comprising the first nucleotide at one or more positions and a stem sequence of the stem loop structure comprises a plurality of nucleotides that is not the first nucleotide. For example, in an extension probe or additional extension probe, the loop comprises the first nucleotide at one or more positions and the stem is made of the other 3 nucleotides, and in an extension reaction performed using a plurality of free nucleotides comprising a terminating nucleotide that base pairs with the first nucleotide, polymerization is halted.

In some embodiments, the terminating group of the terminating nucleotide prevents covalent attachment of another nucleotide during nucleic acid polymerization. Terminating nucleotides can include nucleotides comprising any terminating groups suitable for blocking nucleic acid polymerization. In some embodiments, the terminating nucleotide is a reversible terminator nucleotide. In some examples, the reversible terminator nucleotide comprises an azidomethyl group, an amino group, a nitrobenzyl group, an allyl group, a carbonate, a functional photocleavable ether, a methyl group, or a cyanoethyl group. In some instances, the reversible terminator nucleotide comprises an azidomethyl group, and amino group, a nitrobenzyl group, or an allyl group. In some instances, the reversible terminator nucleotide is a 3′-O-blocked reversible terminator nucleotide. In some embodiments, the terminating nucleotide is an irreversible terminator nucleotide. In some embodiments, the irreversible terminator nucleotide is a 3′ dideoxynucleotide.

In some embodiments, after polymerization is halted at the stopper molecule or stopper modification, the extension probe or additional extension probe is dissociated.

In some embodiments, the detection sequence or amplification sequence comprises a barcode sequence. In some embodiments, a barcode includes two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide. In some embodiments, the methods provided herein includes analyzing the barcodes by sequential hybridization and detection with a plurality of labelled probes (e.g., detection oligos) or by sequencing.

### B. Target RNA Cleavage

In some embodiments, prior to using the extension probe to extend the target RNA, the target RNA is cleaved. In some cases, in order to add a corresponding detection sequence or a complement thereof to a particular target RNA using a polymerase, a particular target sequence at the 3′ end of the target RNA can be needed for hybridizing to a corresponding extension probe. In some cases, the target nucleic acid is an RNA that is cleaved prior to extension. In some cases, the target RNA is cleaved enzymatically, e.g., using an RNase H. In some cases, the target RNA is cleaved using a DNA oligonucleotide, e.g., using a DNAzyme.

In some embodiments, a deoxyribonucleic acid (DNA) oligonucleotide hybridizes to an oligonucleotide hybridization region in a target ribonucleic acid (RNA) and the target RNA is cleaved in the oligonucleotide hybridization region, thereby generating a cleaved target RNA. After cleavage, the extension probe hybridizes to the cleaved target RNA, wherein the extension probe comprises i) a target recognition sequence complementary to a target sequence in the target RNA, and ii) a detection sequence; and the cleaved target RNA is extended with a polymerase using the extension probe as a template, thereby generating an extended nucleic acid molecule. In some embodiments, the target RNA is cleaved enzymatically, e.g., using RNase H. In some embodiments, the target RNA is cleaved by the catalytic DNAzyme. In some embodiments, generating the cleaved target RNA comprises contacting the target RNA with a complex comprising a guide nucleic acid and an RNA-cutting enzyme to guide cutting of a guide target sequence in the target RNA by the RNA-cutting enzyme. In some instances, the RNA-cutting enzyme is an Argonaute protein. In some instances, the RNA-cutting enzyme is a CRISPR effector protein.

In some aspects, the present application provides designs for nucleic acid oligonucleotides capable of forming DNA-RNA duplexes for RNase H cutting in at least a portion of an oligonucleotide hybridization region in a target RNA. In some embodiments, the nucleic acid oligonucleotides is used to direct the cleavage of the target RNA such that the extension probe can bind to the cleaved target RNA for subsequent extension. In some examples, nucleic acid oligonucleotides are designed to hybridize to oligonucleotide hybridization regions having an 8-10 nucleotide overlap with a target sequence for the extension probe to hybridize to the 3′ end of the target RNA.

In some embodiments, the oligonucleotide hybridization region and the target sequence overlap by about 1 to about 20, about 1 to about 15, about 1 to about 10, about 2 to about 10, about 2 to about 9, about 2 to about 8, about 3 to about 15, about 3 to about 10, about 5 to about 15, about 5 to about 10, about 8 to about 12 nucleotides or about 8 to about 10 nucleotides. In some embodiments, the oligonucleotide hybridization region and the target sequence overlap by at least 6 nucleotides, at least 7 nucleotides, 8 nucleotides, at least 9 nucleotides, or at least 10 nucleotides. In some embodiments, the oligonucleotide hybridization region and the target sequence overlap by no more than 15 nucleotides, no more than 12 nucleotides, or no more than 10 nucleotides. In some embodiments, the oligonucleotide hybridization region and the target sequence overlap by about 6 to 10 nucleotides. In some embodiments, the oligonucleotide hybridization region and the target sequence overlap by about 8 to 10 nucleotides. In some embodiments, the oligonucleotide hybridization region and the target sequence overlap by 8 nucleotides. In some embodiments, the oligonucleotide hybridization region and the target sequence overlap by 6 nucleotides. In some examples, the oligonucleotide hybridization region and the target sequence overlap is such that the cutting position is around 6-8 nucleotides from the (3′) end of oligonucleotide. In some examples, the cutting position is between 7-9 nucleotides from the (3′) end of oligonucleotide. In some examples, the cutting position is between 6-7 nucleotides from the (3′) end of oligonucleotide. In some embodiments, the oligonucleotide hybridization region and the target sequence overlap is such that the cutting position is at the 3′ end of the subsequently bound extension probe. In some embodiments, the melting temperature Tₘ of the nucleic acid oligonucleotide about 60° C. to about 70° C., about 65° C. to about 70° C., about 60° C. to about 75° C., about 65° C. to about 68° C. In some embodiments, the melting temperature Tₘ of the oligonucleotide is about 65° C. In some embodiments, the melting temperature Tₘ of the oligonucleotide is at least 65° C. In some embodiments, the melting temperature Tₘ of the oligonucleotide is at least 70° C.

In some embodiments, the nucleic acid oligonucleotide is single-stranded. In some embodiments, the nucleic acid oligonucleotide comprises at least 4, 5, 6, 7, or 8 consecutive deoxyribonucleotides. In some embodiments, the nucleic acid oligonucleotide is a deoxyribonucleic acid (DNA) oligonucleotide. In some cases, the nucleic acid oligonucleotide is a single-stranded DNA (ssDNA) oligonucleotide. In some aspects, the nucleic acid oligonucleotide is a deoxyribozyme (DNAzyme). In some aspects, the nucleic acid oligonucleotide is a catalytic DNAzyme (e.g., a RNA-cleaving DNAzyme).

In some embodiments, the nucleic acid oligonucleotide is designed to hybridize to an oligonucleotide hybridization region comprising a sequence motif for cutting by an RNase H.

In some embodiments, the oligonucleotide hybridization region is about 10 to about 35, about 10 to about 30, about 15 to about 30, about 15 to about 35, about 20 to about 35, about 25 to about 35, about 5 to about 25, about 10 to about 20, about 15 to about 25, about 5 to about 15, about 8 to about 18, about 10 to about 18, or about 15 to about 20 nucleotides in length. In some embodiments, the oligonucleotide is about 10 to about 30, about 15 to about 30, about 5 to about 25, about 10 to about 20, about 15 to about 25, about 5 to about 15, about 8 to about 18, about 10 to about 18, or about 15 to about 20 nucleotides in length. In some embodiments, the oligonucleotide is about 20 to about 35 nucleotides in length. In some embodiments, the oligonucleotide is about 20 to about 30 nucleotides in length. In some embodiments, the length of the nucleic acid oligonucleotide is about 10 to about 40, about 10 to about 35, about 10 to about 30, about 20 to about 40, about 20 to about 35, about 20 to about 30, or about 20 to about 25 nucleotides. In some embodiments, the length of the nucleic acid oligonucleotide is about 20 to about 34 nucleotides. In some embodiments, the length of the nucleic acid oligonucleotide is at least 15 nucleotides, at least 16 nucleotides, 17 nucleotides, at least 18 nucleotides, 19 nucleotides or at least 20 nucleotides.

In some aspects, the nucleic acid oligonucleotide is designed such that upon hybridization of the nucleic acid oligonucleotide to the oligonucleotide hybridization region, the RNase H cleaves the target RNA at a position 5-8 nucleotides from the 3′ end of the hybridized nucleic acid oligonucleotide. In some aspects, the nucleic acid oligonucleotide is designed such that upon hybridization of the nucleic acid oligonucleotide to the oligonucleotide hybridization region, the target RNA is cleaved at a position 5-8 nucleotides from the 3′ end of the hybridized nucleic acid oligonucleotide. In some aspects, the nucleic acid oligonucleotide is designed such that upon hybridization of the nucleic acid oligonucleotide to the oligonucleotide hybridization region, the RNase H cleaves the target RNA at a position 6-7 nucleotides from the 3′ end of the hybridized nucleic acid oligonucleotide.

In some embodiments, the extension probe is added at the same time or after RNA cleavage. In some embodiments, the extension probe is added after generating a cleaved target RNA. In some embodiments, the biological sample is contacted with the DNA oligonucleotide and with the RNase H simultaneously or sequentially (in either order) before contacting the sample with the extension probe. In some embodiments, the biological sample is contacted with the oligonucleotide and with the reagents for cleaving the target RNA (e.g., RNase H, DNAzyme, or a complex comprising a guide nucleic acid and an RNA-cutting enzyme to guide cutting of a guide target sequence in a target ribonucleic acid (RNA) by the RNA-cutting enzyme) before contacting the sample with the extension probe. In some embodiments, the method comprises washing the biological sample after contacting the biological sample with the reagents for cleaving the target RNA and before contacting the biological sample with the extension probe. In some embodiments, RNase inactivating agents or inhibitors can be added to the sample after cleaving the target RNA. In some embodiments, the oligonucleotide hybridization region and the target sequence overlap by about 1 to about 20 nucleotides or by about 8 to about 10 nucleotides.

Provided herein is a method of nucleic acid processing, comprising generating a cleaved target RNA; hybridizing an extension probe to the cleaved target RNA, wherein the extension probe comprises i) a target recognition sequence complementary to a target sequence in the target RNA, and ii) a detection sequence; and extending the cleaved target RNA with a polymerase using the extension probe as a template, thereby generating an extended nucleic acid molecule. In some embodiments, generating the cleaved target RNA comprises hybridizing a deoxyribonucleic acid (DNA) oligonucleotide to an oligonucleotide hybridization region in a target ribonucleic acid (RNA) and cleaving the target RNA in the oligonucleotide hybridization region, thereby generating a cleaved target RNA. In some embodiments, the oligonucleotide hybridization region and the target sequence overlap by about 8 to about 10 nucleotides. In some embodiments, the oligonucleotide hybridization region and the target sequence overlap by about 8 to about 10 nucleotides. In some embodiments, the target sequence is between about 20 and about 60, between about 20 and 50, or between about 30 and about 45 nucleotides in length. In some embodiments, the target sequence is about 25, 30, 35, 40, or 45 nucleotides in length, or any length in a range having endpoints selected from the group consisting of 25, 30, 35, 40, or 45 nucleotides in length. In some embodiments, the oligonucleotide hybridization region is between about 10 and about 30, between about 10 and about 25, between about 15 and about 30, between about 15 and about 25, between about 10 and about 20, or between about 15 and about 25 nucleotides in length.

In some embodiments, provided herein is a method of analyzing a biological sample, comprising: (a) contacting the biological sample with plurality of nucleic acid oligonucleotides, wherein a first oligonucleotide of the plurality hybridizes to a first oligonucleotide hybridization region in a first target ribonucleic acid (RNA) in the biological sample, and a second oligonucleotide of the plurality hybridizes to a second oligonucleotide hybridization region in a second target RNA in the biological sample; (b) contacting the biological sample with an RNase H, wherein the RNase H cleaves the first and second target RNAs in their respective oligonucleotide hybridization regions; (c) contacting the biological sample with a plurality of extension probes, wherein a first extension probe of the plurality comprises a first target recognition sequence complementary to a first target sequence in the first target RNA, wherein a second extension probe of the plurality comprises a second target recognition sequence complementary to a second target sequence in the second target RNA, wherein the first and second extension probes hybridize to their respective target RNAs; extending the cleaved first and second target RNAs each with a polymerase using the first and second extension probes, respectively, as a template, thereby generating an a first and second extended nucleic acid molecule. In some embodiments, the first and second extended nucleic acid molecules are detected. In some embodiments, the first and second extended nucleic acid molecules are further extended using a plurality of amplification probes to generate a first and second extended concatemer. In some embodiments, the first and second extended concatemers are detected.

In some embodiments, the method comprises contacting the biological sample with an RNase H to cleave the target RNA. Any suitable RNase H for cleaving RNA in an nucleic acid duplex (e.g., within the oligonucleotide hybridization region hybridized to the nucleic acid oligonucleotide) can be used. The RNase H enzyme and its family of enzymes include two classes, type 1 and type 2 RNase H based on the difference in their amino acid sequence. Type 1 RNases H include prokaryotic and eukaryotic RNases H1 and retroviral RNase H. Type 2 RNases H include prokaryotic and eukaryotic RNases H2 and bacterial RNase H3. These RNases H exist in a monomeric form, except for eukaryotic RNases H2, which exist in a heterotrimeric form. All of these enzymes share the characteristic that they are able to cleave the RNA component of an RNA: DNA heteroduplex or within a DNA: DNA duplex containing RNA base(s) within one or both of the strands. The cleaved product yields a free 3′-OH for both classes of RNase H. In some embodiments, the RNase H enzyme comprises RNase or RNase H3. In some embodiments, the RNase is an RNase HII.

In some embodiments, the cleaving is performed at a temperature below 60° C. (e.g. at room temperature or about 20-50° C., 20-30° C., 20-40° C., 25-40° C., 30-40° C., 35-40° C., or 40-50° C.). In some embodiments, the cleaving is performed by incubating the biological sample with RNase H at about 37° C. In some embodiments, the cleaving is performed by incubating the biological sample with RNase H at about 37° C. for a duration of between about 10 minutes and about 2 hours, between about 10-120, between about 10-90, between about 10-60, between about 10-30, between about 20-90, between about 20-60, between about 20-30, or between about 30-60 minutes. In some embodiments, the cleaving is performed by incubating the biological sample with RNase H at about 37° C. for a duration of less than 30 minutes. In some embodiments, the cleaving is performed by incubating the biological sample with RNase H at about 37° C. for a duration of about 20 minutes.

In some embodiments, the provided methods comprise contacting the biological sample with RNase H at a concentration of about at least 1×10⁻⁵, 1×10⁻⁴, 1×10⁻³, 1×10⁻², 1×10⁻¹, 1 U/μL, or higher. In some embodiments, the RNase H concentration is less than 1, 1×10−1, 1×10⁻², 1×10⁻³, 1×10⁻⁴, 1×10⁻⁵ U/μL, or less. In some embodiments, the RNase H concentration is between about 1×10⁻⁵ and about 1×10⁻⁴, between about 1×10⁻⁴ and about 1×10⁻³, between about 1×10⁻³ and about 1×10⁻², between about 1×10⁻² and about 1×10⁻¹, between about 1×10⁻¹ and about 1, between about 1×10⁻⁵ and about 1×10⁻³, between about 1×10⁻⁴ and about 1×10⁻², between about 1×10⁻³ and about 1×10⁻¹ U/μL, or between about 1×10⁻² and about 1 U/μL. In some embodiments, the RNase H concentration is about 1×10⁻⁴, 3×10⁻⁴, 1×10⁻³, 3×10⁻³, 1×10⁻², 3×10⁻², or 1×10⁻¹ U/μL. In some embodiments, the RNase H concentration is about 1×10⁻², 3×10⁻², or 1×10⁻¹ U/μL. In some embodiments, the RNase H concentration is about 1×10⁻² U/μL.

In some embodiments, the provided methods comprise contacting the biological sample with between about 0.5 enzyme units (U) and about 100 U of the RNase H. In some embodiments, the biological sample is contacted with between about 0.5 and about 100, between about 0.5 and about 80, between about 0.5 and about 60, between about 0.5 and about 50, between about 0.5 and about 40, between about 0.5 and about 30, between about 0.5 and about 20, between about 0.5 and about 10, between about 0.5 and about 8, between about 0.5 and about 5, between about 0.5 and about 5, between about 0.5 and about 3, between about 2 and about 10, between about 2 and about 8, between about 2 and about 6, between about 2 and about 5, between about 2 and about 4, between about 3 and about 8, between about 3 and about 6, between about 4 and about 8, between about 4 and about 6, between about 4 and about 5.5, between about 4.5 and about 5.5, between about 4.5 and about 6, between about 10 and about 100, between about 10 and about 50, between about 10 and about 30, between about 10 and about 20, between about 20 and about 100, between about 20 and about 50, between about 20 and about 40, between about 20 and about 30, between about 30 and about 100, between about 30 and about 80, or between about 30 and about 50 U of RNase H. In some embodiments, the amount of RNase H contacted with the biological sample is dependent on the amount of nucleic acid oligonucleotide to be cleaved in the biological sample.

In some embodiments, the RNase His incubated with the biological sample in a buffer comprising magnesium chloride. In some embodiments, the RNase H is incubated with the biological sample in a buffer comprising magnesium chloride, potassium chloride, dithiothreitol (DTT), and a buffering agent (e.g., Tris-HCL).

In some embodiments, the RNase H comprises an RNase H1 and/or an RNase H2. In some embodiments, the method comprises contacting the biological sample with an RNase H1 and an RNase H2.

In some embodiments, the RNase His RNase H1. In some embodiments, the RNase His an endoribonuclease that specifically hydrolyzes the phosphodiester bonds of RNA which is hybridized to DNA. In some embodiments, the RNase H does not digest single or double-stranded DNA. In some embodiments, the RNase H requires at least four contiguous bases of RNA for digestion of the RNA hybridized to DNA. In some embodiments, the RNase H does not digest a di-ribonucleotide-containing DNA sequence (e.g., one that is DNA-annealed). In some embodiments, the RNase H does not digest a mono-ribonucleotide-containing DNA sequence (e.g., one that is DNA-annealed).

In some embodiments, the RNase His RNase H2. In some embodiments, the RNase His an endoribonuclease that preferentially nicks 5′ to one or more ribonucleotides (e.g., a single ribonucleotide, a diribonucleotide sequence, etc.) within the context of a DNA duplex, leaving 5′ phosphate and 3′ hydroxyl ends. In some embodiments, the RNase H nicks at multiple sites along an RNA portion hybridized to a DNA. In some embodiments, the RNase H digests a DNA-annealed di-ribonucleotide-containing DNA sequence, whereas the RNA-annealed di-ribonucleotide-containing DNA sequence is not digested. In some embodiments, the RNase H digests a DNA-annealed mono-ribonucleotide-containing DNA sequence, whereas RNA-annealed mono-ribonucleotide-containing DNA sequence is not digested. In some embodiments, the RNase H digests a target RNA but does not digest an extension probe hybridized to the target RNA, such that the digested target RNA is extendible using the hybridized extension probe as a template.

In some embodiments, the RNase H cleaves RNA in RNA-DNA duplexes. In some embodiments, the RNase His a bacterial RNase H or analog or derivative thereof (e.g., Escherichia coli RNase H or an analog or derivative thereof). In some embodiments, the RNase H exhibits a sequence preference for cleavage, and the oligonucleotide hybridization region is designed to contain a sequence motif for RNase H cleavage.

In some embodiments, the target RNA is cleaved using an RNA-cutting enzyme and a guide nucleic acid to provide a DNA-RNA or RNA duplex upon hybridization to the target RNA, for cutting of the target RNA by the RNA-cutting enzyme. In some embodiments, the method comprises providing a complex comprising a guide nucleic acid and an RNA-cutting enzyme wherein the guide nucleic acid binds to a guide target sequence in a target RNA to form a DNA-RNA or RNA duplex with at least a portion of the guide target sequence. The complex comprising the RNA-cutting enzyme can then cut the target RNA within the guide target sequence. In some instances, the guide target sequence overlaps with the extension probe target sequence. In some embodiments, after cleavage by the RNA-cutting enzyme (e.g., Argonaute protein or CRISPR effector protein, the target sequence for hybridizing to the extension probe is position at the 3′ end of the cleaved target RNA.

In some embodiments, the guide nucleic acid comprises RNA. In some embodiments, the guide nucleic acid comprises DNA. In some embodiments, the guide nucleic acid comprises both DNA and RNA. In some embodiments, the guide nucleic acid is single-stranded. In some cases, the guide nucleic acid is a single-stranded DNA (ssDNA) oligonucleotide.

In some embodiments, the RNA-cutting enzyme is an Argonaute protein. In some embodiments, the Argonaute protein is an RNA-guided Argonaute, and the guide nucleic acid is an RNA molecule. In some embodiments, the Argonaute protein is a DNA-guided Argonaute, and the guide nucleic acid is a DNA molecule. In some embodiments, the guide nucleic acid comprises a 5′-phosphate or a 5′-OH. The guide nucleic acid is at least about 5, at least about 8, at least about 10, at least about 12, at least about 15, at least about 20, or at least about 30 nucleotides in length. In some embodiments, the guide nucleic acid is between about 10 and about 30, about 15 and about 25, about 14 and about 20, about 16 and about 20, about 20 and about 30 nucleotides, or about 25 and about 35 nucleotides in length. In some embodiments, the guide target sequence is at least about 5, at least about 8, at least about 10, at least about 12, at least about 15, at least about 20, or at least about 30 nucleotides in length. In some embodiments, the guide target sequence is between about 10 and about 30, about 15 and about 25, about 14 and about 20, about 16 and about 20, about 20 and about 30 nucleotides, or about 25 and about 35 nucleotides in length. In some embodiments, the guide nucleic acid is fully complementary to the guide target sequence (e.g., hybridizable). In some embodiments, the guide nucleic acid is partially complementary to the guide target sequence. In some embodiments, the guide nucleic acid is at least about 30, about 40, about 50, about 60, about 70, about 80, about 90, about 95, or about 100% complementary to the guide target sequence.

In some embodiments, the RNA-cutting enzyme is a CRISPR effector protein and the guide nucleic acid is a CRISPR guide RNA. In some embodiments, the guide nucleic acid can comprise a spacer sequence, which is a sequence capable of hybridizing to a guide target sequence in a target RNA. In some embodiments, the spacer sequence is located at the 5′ end of the guide nucleic acid. In some embodiments, the spacer sequence is at least about 5, at least about 8, at least about 10, at least about 12, at least about 15, at least about 20, or at least about 30 nucleotides in length. In some embodiments, the spacer sequence is between about 10 and about 30, about 15 and about 25, about 20 and about 30, or about 25 and about 35 nucleotides in length. In some embodiments, the spacer sequence is 20 to 30 nucleotides in length. In some embodiments, the spacer sequence is 28 to 30 nucleotides in length. In some embodiments, the guide target sequence is at least about 5, at least about 8, at least about 10, at least about 12, at least about 15, at least about 20, or at least about 30 nucleotides in length. In some embodiments, the guide target sequence is between about 10 and about 30, about 15 and about 25, about 20 and about 30, or about 25 and about 35 nucleotides in length. In some embodiments, the guide target sequence is 20 to 30 nucleotides in length. In some embodiments, the guide target sequence is 28 to 30 nucleotides in length. In some embodiments, the spacer sequence is fully complementary to the guide target sequence. In some embodiments, the spacer sequence is partially complementary to the guide target sequence. In some embodiments, the spacer sequence is at least about 30, about 40, about 50, about 60, about 70, about 80, about 90, about 95, or about 100% complementary to the guide target sequence. In some embodiments, the guide nucleic acid comprises a scaffold region that binds to the CRISPR effector protein. In some embodiments, the scaffold region is located at the 3′ end of the guide nucleic acid.

In some embodiments, the method comprises contacting the biological sample with a guide nucleic acid and an RNA-cutting enzyme. In some embodiments, the RNA-cutting enzyme is an Argonaute protein. Any suitable Argonaute protein for cutting RNA in a nucleic acid duplex can be used. Generally, Argonaute proteins contain 6 main domains (N-terminal, L1 (Linker 1), PAZ (Piwi-Argonaute-Zwille), L2 (Linker 2), MID (Middle) and PIWI (P-element induced wimpy testis) responsible for binding of a guide nucleic acid and recognition of a guide target sequence. More specifically, the PIWI domain can possess a nuclease active site with a catalytic tetrad (e.g., amino acid sequence DEDX, wherein X is the amino acid D, H, or K), wherein the catalytic tetrad coordinates two divalent metal cations (e.g., Mn²⁺, Mg²⁺, etc.) essential for target cleavage. In some embodiments, the Argonaute protein is an RNA-guided Argonaute, and the guide nucleic acid is an RNA molecule. In some embodiments, the Argonaute protein is a DNA-guided Argonaute, and the guide nucleic acid is a DNA molecule.

In some embodiments, the Argonaute protein is a eukaryotic Argonaute protein. Generally, eukaryotic Argonaute proteins can mediate cutting of a target RNA with a guide nucleic acid of RNA. In some embodiments, an Argonaute protein is of plant, algal, fungal (e.g., yeast), or animal (e.g., human, rodent, fruit fly, cnidarian, echinoderm, nematode, fish, amphibian, reptile, bird, etc.) origin. In some embodiments, the Argonaute protein is Ago1, Ago2, Ago3, Ago4, PIWI 1, PIWIL 2, PIWI 3, or PIWI 4. In some embodiments, the Argonaute protein is Ago2. In some embodiments, the Ago2 is Drosophila Ago2. In some embodiments, the Argonaute protein is a recombinant Drosophila Argonaute protein. In some embodiments, the Argonaute protein is expressed in a mammalian cell line. In some embodiments, the Argonaute protein is a Drosophila Argonaute protein expressed in a mammalian cell line. In some embodiments, a Drosophila Argonaute protein is expressed using a method such that a loading complex specific to Drosophila species is not provided to obtain guide-free proteins. In some embodiments, the Argonaute protein is a purified recombinant Drosophila Argonaute protein. In some embodiments, the Argonaute protein is expressed in an insect cell line, such as a Schneider 2 (S2) cell line. In some embodiments, the Argonaute protein is a Drosophila Argonaute protein expressed in an insect cell line, such as a S2 cell line. In some embodiments, the Drosophila Argonaute protein is loaded with the guide nucleic acid prior to contacting the biological sample. In some embodiments, the Argonaute protein is from Thermomyces thermophilus. In some embodiments, an Argonaute protein is from Vanderwaltozyma polyspora (also known as Kluyveromyces polysporus) (such as an Argonaute protein described in WO 2018/112336, the content of which is herein incorporated by reference in its entirety).

In some embodiments, the method comprises contacting the biological sample with a guide nucleic acid and an RNA-cutting enzyme. In some embodiments, the RNA-cutting enzyme is a CRISPR effector protein. Generally, a CRISPR effector protein can form a complex with a guide nucleic acid, and the complex functions as a CRISPR-Cas system. In some embodiments, the guide nucleic acid is a CRISPR guide RNA comprising a spacer sequence, wherein the spacer sequence hybridizes to the guide target sequence. Any suitable CRISPR-Cas systems can be used for cutting RNA in a nucleic acid duplex, and example Cas effector proteins are described in herein.

In general, a CRISPR-Cas system is characterized by elements that promote the formation of a CRISPR complex at the site of a target RNA sequence (also referred to as a protospacer in the context of an endogenous CRISPR system). CRISPR-Cas systems form two major classes that differ in the organization of their effector modules. In Class 1 systems, multiple protein units form an effector complex together with the CRISPR RNA (crRNA) to recognize and cut a target RNA sequence, whereas a single protein complexing with crRNA does the job in a Class 2 system. To date, there are six types of CRISPR-Cas systems discovered: type I, type III, and type IV are identified as Class 1 systems, while type II, type V, and type VI are classified as Class 2. The specificity of cutting in CRISPR-Cas systems is conferred by RNA-based guidance through base-pairing, and the guide sequences can be adjusted to cut a new sequence.

In some embodiments, the CRISPR effector protein is a Class 2, Type VI Cas protein. In some embodiments, the CRISPR effector protein is a Class 2, Type II Cas protein. In some embodiments the CRISPR effector protein is a Cas13 protein. In some embodiments the CRISPR effector protein is a Cas9 protein.

In some embodiments, the RNA targeting Cas protein is a Cas9 protein, which in some instances is referred to as RNA-targeting Cas9 (RCas9). In some embodiments, the Cas9 protein comprises a mutation in the naturally occurring Cas9. In some embodiments, a Cas9 protein is engineered to target RNA instead of DNA. In some embodiments, an engineered nucleoprotein complex comprises a Cas9 protein and a single guide RNA (sgRNA) to recognize a target RNA sequence. Optionally, in such systems, an (chemically-modified or synthetic) antisense PAMmer oligonucleotide can be included to simulate a DNA substrate for recognition by Cas9 via hybridization to the target RNA. In some embodiments, the Cas9 protein is a S. pyogenes Cas9 (SpyCas9) and the method comprises contacting the biological sample with a DNA oligonucleotide comprising the cognate PAM sequence (a PAMmer).

In some embodiments, the Cas9 as provided herein is further complexed with an antisense guide oligonucleotide which is complementary to a sequence in the target RNA. In some embodiments, the antisense guide oligonucleotide comprises a PAMmer oligonucleotide. In some embodiments, the antisense guide oligonucleotide comprises at least one modified nucleotide. In some embodiments, the at least one modified nucleotide is selected from the group consisting of 2′OMe RNA and 2′OMe DNA nucleotides. In some embodiments, the PAMmer oligonucleotide comprises one or more modified bases or linkages. In some embodiments, the one or more modified bases or linkages are selected from the group consisting of locked nucleic acids and nuclease stabilized linkages. In some embodiments, the antisense guide oligonucleotide is complementary to a sequence that is in close proximity to the target RNA. For example, the antisense guide oligonucleotide can be complementary to a sequence that is about 10 nt, 20 nt, 30 nt, 40 nt, 50 nt, 60 nt, 70 nt, 80 nt, 90 nt, 100 nt, from the target RNA. In some embodiments, the antisense guide oligonucleotide has a length that is about, is less than, or is more than, 10 nt, 20 nt, 30 nt, 40 nt, 50 nt, 60 nt, 70 nt, 80 nt, 90 nt, 100 nt, 110 nt, 120 nt, 130 nt, 140 nt, 150 nt, 160 nt, 170 nt, 180 nt, 190 nt, 200 nt, 300 nt, 400 nt, 500 nt, 1,000 nt, 2,000 nt, or a range between any two of the above values. In some embodiments the antisense guide oligonucleotide comprises RNA, DNA, or both.

In some embodiments, the guide nucleic acid is designed such that upon hybridization of the spacer sequence to the guide target sequence, the CRISPR effector protein cuts the target RNA at a position about 3 to about 15, about 5 to about 8, about 8 to about 15, about 12 to about 20, about 15 to about 25, or about 25 to about 35 nucleotides from the 3′ end of the hybridized spacer sequence.

### C. Target RNA Extension

In some embodiments, the target RNA is extended by a polymerase using the extension probe as a template. In some aspects, the extension of the target RNA generates an extended concatemer that is an amplification product. In some embodiments, a sequence corresponding to the target RNA is amplified in the generated extended concatemer. The methods for target extended amplification provided herein can be used to detect and/or analyze one or more target RNAs (e.g., nucleic acid analytes). Examples of nucleic acid analytes include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). In some embodiments, the RNA and the generated extended concatemer is single-stranded. In some instances, the target RNA is a microRNA (miRNA). In some embodiments, the target RNA is not mRNA.

Methods and compositions disclosed herein can be used to analyze any number of target RNAs. For example, the number of target RNAs that are analyzed using the extension methods disclosed herein can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000 or more different target RNAs present in a region of the biological sample. In some instances, the number of target RNAs that are analyzed is at least about 100, at least about 500, at least about 1,000, at least about 10,000 or more different target RNAs present in a region of the biological sample, the number of target RNAs that are extended by a polymerase is at least about 100, at least about 500, at least about 1,000 or more different target RNAs present in a region of the biological sample. In some embodiments, the extension of a plurality of RNAs occurs in the same reaction mixture. In some embodiments, the extension of a plurality of different RNAs is performed using a plurality of different extension probes.

In any embodiment described herein, the target RNA (e.g., an mRNA) comprises a target sequence for a corresponding extension probe. In some embodiments, the target sequence is endogenous to the sample. In some embodiments, the target sequence is a single-stranded target sequence in the target RNA. In some embodiments, the target sequence is uniquely associated with the target RNA. In some embodiments, the target sequence is unique to the target RNA among the different target RNAs present in the biological sample, or among the target RNAs detectably expressed in the biological sample. In some embodiments, the target sequence uniquely identifies the gene encoding the target RNA among the detectably expressed genes in the biological sample. In some embodiments, a target RNA or each target RNA comprises a single target sequence. In some embodiments, a first target RNA comprises a first target sequence, a second target RNA comprises a second target sequence, and an Nth target RNA comprises an Nth target sequence, wherein the first, second, and Nth target sequence are different. In some instances, the target sequence is not a poly A sequence. In some instances, the target recognition sequence is not a poly T sequence.

In some embodiments, the target RNA(s) is/are attached directly or indirectly to the biological sample or to a matrix embedding the biological sample. In some embodiments, the target RNA(s) is/are crosslinked in the biological sample or in a matrix embedding the biological sample. In some embodiments, the generated extension product (e.g., extended nucleic acid molecule or extended concatemer) is covalently linked to the cleaved target RNA or a portion thereof. In some examples, the polymer matrix can be a hydrogel. In some embodiments, cross-linking of the matrix or components to be anchored to the matrix are performed chemically and/or photochemically, or alternatively by any other suitable hydrogel-formation method.

In some embodiments, performing the extension of the target RNA or an extension thereof comprises incubating the biological sample with a polymerase for a duration of between about 10 minutes and about 4 hours, between about 10-120 minutes, between about 30-120 minutes, between about 20-90 minutes, between about 60-90 minutes, between about 30-90 minutes, between about 30-60 minutes, between about 60-120 minutes, or between about 60-135 minutes. In some embodiments, performing the extension of the target RNA or an extension thereof comprises incubating the biological sample at a temperature between about 20° C. and about 60° C. In some embodiments, performing the extension of the target RNA or an extension thereof comprises incubating the biological sample with a polymerase for about 30 minutes at about 30-40° C. (e.g., at about 37° C.). In some embodiments, performing the extension of the target RNA or an extension thereof comprises incubating the biological sample with a polymerase for about 1 hour at about 30-40° C. (e.g., at about 37° C.). In some embodiments, performing the extension of the target RNA or an extension thereof comprises incubating the biological sample with a polymerase for about 2 hours minutes at about 30-40° C. (e.g., at about 37° C.). In some embodiments, performing the extension of the target RNA or an extension thereof comprises incubating the biological sample with a polymerase for about 30 minutes at about 40-50° C. (e.g., at about 45° C.). In some embodiments, performing the extension of the target RNA or an extension thereof comprises incubating the biological sample with a polymerase for about 1 hour at about 40-50° C. (e.g., at about 45° C.).

In some embodiments, the polymerase is a strand-displacing polymerase. In some embodiments, the polymerase is selected from phi29 DNA polymerases, Bst DNA polymerases, and Bsu DNA polymerase, large fragment. In some embodiments, the polymerase is a Phi29 DNA polymerase, Phi29-like DNA polymerase, M2 DNA polymerase, B103 DNA polymerase, GA-1 DNA polymerase, phi-PRD1 polymerase, Vent DNA polymerase, Deep Vent DNA polymerase, Vent (exo-) DNA polymerase, KlenTaq DNA polymerase, DNA polymerase I, Klenow fragment of DNA polymerase I, DNA polymerase III, T3 DNA polymerase, T4 DNA polymerase, T5 DNA polymerase, T7 DNA polymerase, Bst polymerase, rBST DNA polymerase, N29 DNA polymerase, TopoTaq DNA polymerase, T7 RNA polymerase, SP6 RNA polymerase, T3 RNA polymerase, or a variant or derivative of any of the foregoing polymerases. In some embodiments, the polymerase is a Phi29 polymerase. In some instances, the polymerase for extension is a phi29 DNA polymerase, a Bst DNA polymerase, or a Bsu DNA polymerase, or a variant or derivative of any of the foregoing polymerases.

In some aspects, the extended nucleic acid molecule comprises the detection sequence or a complement thereof and at least two copies of the amplification sequence or a complement thereof. In some aspects, the extended concatemer comprises the detection sequence or a complement thereof and at least two copies of the amplification sequence or a complement thereof. In some embodiments, the generated concatemer comprises at least 20, at least 30, or at least 40 nucleotides of the target RNA. In some aspects, the target RNA or portion thereof, the detection sequence or a complement thereof and at least two copies of the amplification sequence or a complement thereof in the extended concatemer are covalently attached within the one nucleic acid molecule.

### D. Detection and Analysis

In some embodiments, a method disclosed herein comprises detecting one or more extension products of the target nucleic acids (e.g., target RNA) in a sample. In some embodiments, a method disclosed herein comprises detecting a plurality of generated extension products of the target nucleic acids (e.g., target RNA) in a sample. In some embodiments, the detection sequence of a portion or a complement thereof in an extended nucleic acid molecule is detected. In some embodiments, the amplification sequence or a portion or a complement thereof in an extended concatemer is detected. In some embodiments, the detection sequence comprises a barcode sequence. In some embodiments, the amplification sequence comprises a barcode sequence. In some aspects, the generated extended concatemer comprises two or more different barcode sequences. In some embodiments, the detection may be spatial, e.g., in two or three dimensions. In some instances, the target RNA is in a biological sample. In some instances, the extended nucleic acid molecule or the extended concatemer is analyzed at a location in the biological sample or a matrix embedding the biological sample. In some embodiments, the biological sample is a fixed and/or permeabilized biological sample. In some instances, wherein the biological sample is a cell or tissue sample. In some embodiments, the extension is performed at a spatial location on a solid substrate having the biological sample attached thereto.

In some aspects, the extension product (e.g., the extended nucleic acid molecule or the extended concatemer) is a single continuous nucleic acid molecule comprising the target RNA (or a portion thereof), the detection sequence or a complement thereof, and two or more copies of the amplification sequences or complements thereof. In some instances, the extended concatemer is not cleaved prior to detecting the detection sequence and/or a complement thereof. In some instances, the extended concatemer is not cleaved prior to detecting the amplification sequence and/or a complement thereof. In some instances, sequences of an intact extended concatemer is detected. In some instances, the extended nucleic acid molecule comprises a cleavage site. In some instances, the extended concatemer comprises a cleavage site. For example, the cleavage site is a restriction digestion cleavage site recognized by a restriction enzyme. In some cases, the cleavage site is recognized by a DNAzyme. In some cases, the method comprises cleaving the extended concatemer using a DNA oligonucleotide (e.g., a DNAzyme). In some aspects, cleaving the extended concatemer comprises chemical cleavage. In some aspects, the extended concatemer comprises a disulfide bond. In some aspects, the chemical cleavage is performed using a reducing agent. In some instances, the reducing agent is DTT or THPP. In some instances, the extended concatemer is cleaved prior to detection.

In some embodiments, the detection may be quantitative, e.g., the amount or concentration of a target nucleic acid may be determined. In some embodiments, the extension probes or additional extension probes may comprise any of a variety of entities able to hybridize a nucleic acid, e.g., DNA, RNA, LNA, and/or PNA, etc., depending on the application.

In some embodiments, detecting the extension products of the target nucleic acids (e.g., target RNA) comprises sequential hybridization cycles of detectably labeled probes that directly or indirectly hybridize to detection sequences or amplification sequences or complements thereof (e.g., barcode sequences) or subunits thereof in the extension products (e.g., in the generated extended concatemer). In some embodiments, detecting the extension products of the target nucleic acids (e.g., target RNA) comprises sequential hybridization cycles of intermediate probes that hybridize to barcode sequences or subunits thereof in the extension products, and detectably labeled probes that bind directly or indirectly to the intermediate probes. For example, in some embodiments, detecting the barcode sequences or complements thereof in the extension products comprises: contacting the test biological sample with a universal pool of detectably labeled probes and a first pool of intermediate probes, wherein the intermediate probes of the first pool of intermediate probes comprise hybridization regions complementary to the barcode sequence or complements thereof and reporter regions complementary to a detectably labeled probe of the universal pool of detectably labeled probes; detecting complexes formed between the barcode sequences or complements thereof, the intermediate probes of the first pool of intermediate probes, and the detectably labeled probes; and removing the intermediate probes of the first pool of intermediate probes and the detectably labeled probes. In some embodiments, detecting the barcode sequences or complements thereof further comprises: contacting the test biological sample with the universal pool of detectably labeled probes and a second pool of intermediate probes, wherein the intermediate probes of the second pool of intermediate probes comprise hybridization regions complementary to the barcode sequences or complements thereof and reporter regions complementary to a detectably labeled probe of the universal pool of detectably labeled probes; and detecting complexes formed between the barcode sequences or complements thereof, the intermediate probes of the second pool of intermediate probes, and the detectably labeled probes. In some embodiments, each barcode sequence or complement thereof is assigned a series of signal codes that identifies the barcode sequence or complement thereof, and detecting the barcode sequences or complements thereof comprises decoding the barcode sequences of complements thereof by detecting the corresponding sequences of signal codes detected from sequential hybridization, detection, and removal of sequential pools of intermediate probes and the universal pool of detectably labeled probes. In some embodiments, the series of signal codes are fluorophore sequences assigned to the corresponding target RNA.

In some embodiments, detecting the extension products of the target nucleic acids (e.g., target RNA) comprises detecting a series of barcode sequences (e.g., subunits of a barcode sequence that together identify the corresponding target RNA). In some embodiments, detecting the series of barcode sequences comprises sequential hybridization of probes to different barcode sequences or subunits present in an extension product of the target nucleic acids (e.g., target RNA) in a pre-determined order. For example, a first detectably labeled probe can be hybridized to a first barcode sequence or barcode subunit in an extension product and detected (e.g., by imaging the biological sample). After detection of the first detectably labeled probe, the probe can be removed by washing, or a detectable label associated with the first detectably labeled probe can be quenched or removed by cleavage (e.g., cleavage of a disulfide linker connecting the detectable label to the probe). Next, a second detectably labeled probe can be hybridized to a second barcode sequence or barcode subunit in the extension product and detected (e.g., by imaging the biological sample). In some cases, the extension product is assigned a series of signal codes that identify the corresponding target RNA. For example, in some embodiments, the first detected signal for the first detectably labeled probe hybridized to the first barcode sequence or barcode subunit corresponds to the first signal code in the series, and the second detected signal for the second detectably labeled probe hybridized to the second barcode sequence or barcode subunit corresponds to the second signal code in the series. In some embodiments, the series of signal codes are fluorophore sequences assigned to the corresponding target RNA.

In some embodiments, the sample is contacted with a plurality of detectable probes, wherein each detectable probe is configured to hybridize to a complement of a detection sequence or an amplification sequence introduced by polymerization using the extension probe or the additional extension probe. In some embodiments, a barcode sequence or complement thereof is present in multiple copies in a generated extension product, e.g., an extended nucleic acid molecule or an extended concatemer comprising the target RNA or a portion thereof. In some embodiments, the method further comprises detecting a signal associated with the plurality of detectable probes or absence thereof at one or more locations in the sample. In some embodiments, the sample is contacted with a subsequent plurality of detectable probes, wherein each detectable probe in the subsequent plurality is configured to hybridize to the extension product directly or indirectly, e.g., an extended nucleic acid molecule or an extended concatemer. In some embodiments, the detection sequence or a complement thereof is present in multiple copies in the generated extension product, e.g., an extended nucleic acid molecule or an extended concatemer. In some embodiments, the amplification sequence or a complement thereof is present in multiple copies in the generated extension product, e.g., an extended nucleic acid molecule or an extended concatemer. In some embodiments, the method further comprises detecting a subsequent signal associated with the subsequent plurality of detectable probes or absence thereof at the one or more locations in the sample. In some embodiments, the method further comprises generating a signal code sequence comprising signal codes corresponding to the signal or absence thereof and the subsequent signal or absence thereof, respectively, at the one or more locations, wherein the signal code sequence corresponds to one of the one or more target nucleic acids, thereby identifying the target nucleic acid at the one or more locations in the sample. In some embodiments, the extension of multiple target nucleic acids (e.g., target RNA) are performed at a plurality of locations in the sample. In some aspects, the extension of multiple target nucleic acids (e.g., target RNA) are generated using fragments of the cleaved target RNAs.

In some embodiments, provided herein are methods, compositions, kits, and systems for performing an extension reaction of a target nucleic acid associated with a non-nucleic acid analyte. In some aspects, an assay is performed to detect target RNAs and non-nucleic acid analytes using the extension reactions described herein. In some embodiments, provided herein are methods and compositions for analyzing endogenous non-nucleic acid analytes (e.g., cell surface or intracellular proteins, and/or metabolites) in a sample using one or more labeling agents. In some embodiments, an analyte labeling agent may include an agent that interacts with an analyte (e.g., an endogenous analyte in a sample). In some embodiments, the labeling agent can comprise a reporter oligonucleotide that is indicative of the analyte or portion thereof interacting with the labeling agent. For example, the reporter oligonucleotide may comprise a barcode sequence that permits identification of the labeling agent. In some embodiments, the analyte labeling agent comprises an analyte binding moiety and a labeling agent barcode domain comprising one or more barcode sequences, e.g., a barcode sequence that corresponds to the analyte binding moiety and/or the analyte.

In some aspects, the reporter oligonucleotide comprised by the labeling agent is extended to generate an amplification product. For example, a generated extension product is an extended nucleic acid molecule or an extended concatemer comprising the reporter oligonucleotide and one or more additional sequences added by extension. In some embodiments, the one or more additional sequences added by extension comprises a detection sequence. In some embodiments, the one or more additional sequences added by extension comprises an amplification sequence. In some embodiments, the one or more additional sequences added by extension comprises two or more copies of a barcode sequence corresponding to the labeling agent. In some aspects, the extension is performed using an extension probe and optionally, one or more additional extension probes (e.g., as described in Section II.A). In some aspects, the extension adds a detection sequence to the reporter oligonucleotide. In some aspects, the extension adds an amplification sequence to the reporter oligonucleotide. In some aspects, the methods provided herein generate a first plurality of amplification products from extension of target RNAs and a second plurality of amplification products from extension of reporter oligonucleotides comprised by labeling agents. In some aspects, the extension of the target RNA and the reporter oligonucleotide can be tuned to achieve a desired level of amplification (e.g., by controlling the concentration of extension probes and additional extension probes provided for the extension reaction). In some aspects, generating amplification products from extension of reporter oligonucleotides comprised by labeling agents may allow for bright signals to be detected associated with the labeling agents to identify the associated non-nucleic acid analyte. In some aspects, the extension of reporter oligonucleotides comprised by labeling agents using extension probes and optionally one or more additional extension probes is a fast reaction (e.g., no more than 30 minutes for amplification). In some aspects, generating amplification products from extension of reporter oligonucleotides comprised by labeling agents may allow for direct detection of the generated amplification products (e.g., without the use of intermediate probes). In some aspects, generating amplification products from extension of reporter oligonucleotides comprised by labeling agents may allow for the reporter oligonucleotide conjugated to the labeling agent to be a shorter length compared to an alternative labeling agent detection method that does not utilize extension probes for amplification.

In some embodiments, the method comprises one or more post-fixing (also referred to as post-fixation) operations after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of example labeling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or epitope-binding fragments thereof. The antibodies or epitope-binding fragments including the analyte binding moiety can specifically bind to a target analyte. In some embodiments, the analyte is a protein (e.g., a protein on a surface of a cell) or an intracellular protein). In some embodiments, a plurality of analyte labeling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labeling agents are the same. In some embodiments in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

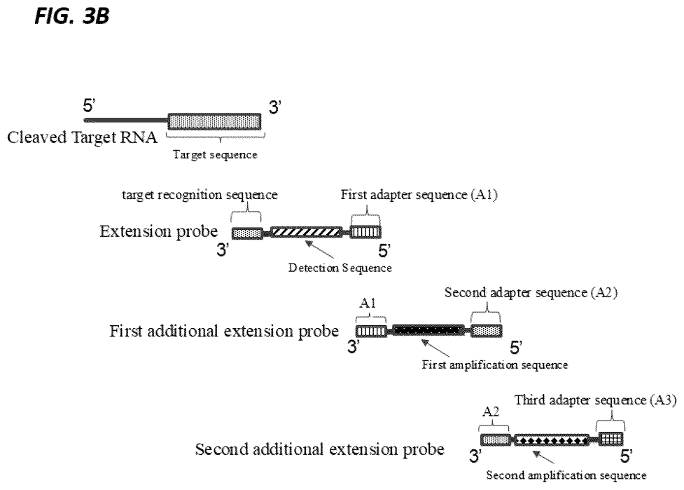

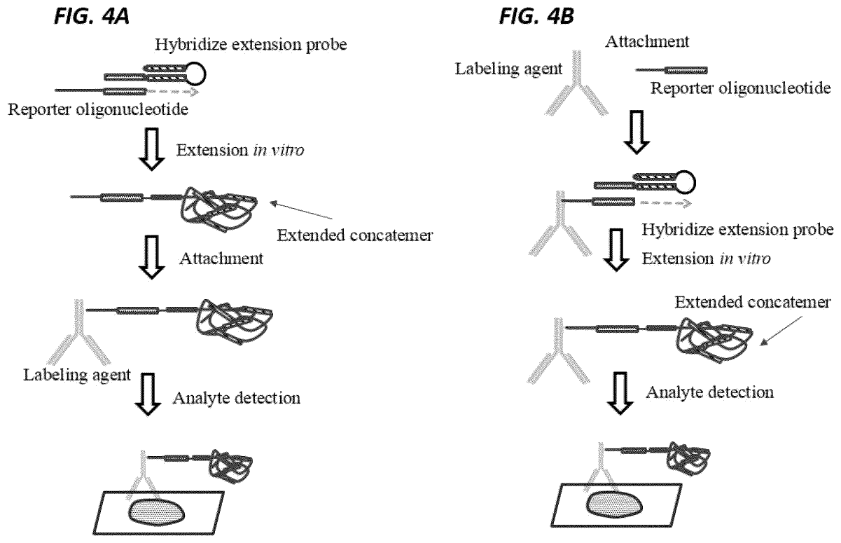

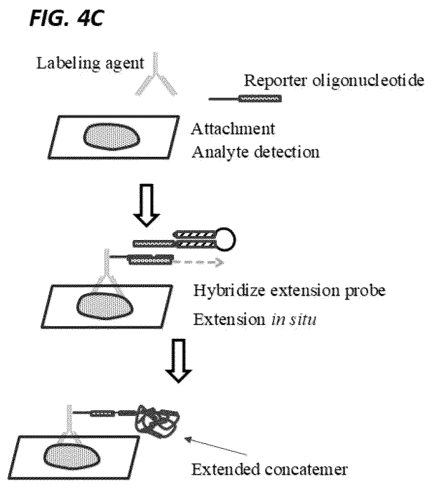

In some aspects, the reporter oligonucleotide comprises nucleic acid barcode sequence(s) that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using the in situ detection techniques described herein. In some embodiments, a reporter oligonucleotide is extended to generate a concatemer prior to attachment to the labeling agent (e.g., as shown in FIG. 4A). In some embodiments, a reporter oligonucleotide is extended to generate a concatemer after attachment to the labeling agent (e.g., as shown in FIGS. 4B and 4C). In some embodiments, a reporter oligonucleotide is extended to generate a concatemer prior to contacting the labeling agent to the biological sample (e.g., as shown in FIG. 4A and FIG. 4B). In some embodiments, a reporter oligonucleotide is extended to generate a concatemer after contacting the labeling agent to the biological sample (e.g., as shown in FIG. 4C).

In some instances, the extension of a reporter oligonucleotide is performed using an extension probe (e.g., as described in Section II) as template. In some cases, the generated extended nucleic acid molecule comprises a detection sequence or a complement thereof. In some aspects, the generated extended nucleic acid molecule is further extended by a polymerase to add one or more additional amplification sequence(s) or complements thereof. In some cases, the generated extended concatemer generated by extending the reporter oligonucleotide is covalently attached to their respective labeling agent, as illustrated in FIG. 4A-4C. In FIG. 4A-4C, the extended probe as illustrated and optionally additional extended probes may have a stem loop structure or may be linear oligonucleotides. In some aspects, the generated extended nucleic acid molecule comprises a complement of the detection sequence from the extension probe used as a template. In some aspects, the generated extended nucleic acid molecule comprises at least two copies of the detection sequence (or a complement thereof) from the extension probe used as a template. In some aspects, the detection sequence is a barcode sequence corresponding to the analyte bound by the labeling agent. In some aspects, the generated extended nucleic acid molecule is further extended using a plurality of additional extension probes. In some aspects, an additional extension probe comprises at least a portion of the detection sequence of the extension probe or a complement thereof, and an amplification sequence. An additional extension probe serves as template to further extend the extended nucleic acid molecule to generate an extended concatemer which comprises the reporter oligonucleotide, the detection sequence or a complement thereof and the amplification sequence or a complement thereof. In some instances, the generated extended concatemer comprises at least two copies of the amplification sequence or complements thereof.

In some embodiments, an extension probe comprises a target recognition sequence that is complementary to a target sequence in the reporter oligonucleotide. In some instances, the target recognition sequence of the extension probe may be of any length, and multiple recognition sequences in the same or different extension probes may be of the same or different lengths. For instance, the target recognition sequence may be at least 20, at least 25, at least 30, at least 35, at least 40, or at least 50 nucleotides in length. In some embodiments, the target recognition sequence may be no more than 48, no more than 45, or no more than 40 nucleotides in length. Combinations of any of these are also possible, e.g., the recognition sequence may have a length of between 25 and 40, between 30 and 45, or between 20 and 48 nucleotides, etc. In some embodiments, the target recognition sequence is at least 95%, at least 98%, at least 99%, or at least 100% complementary to the target sequence in the reporter oligonucleotide.

In some embodiments, the target recognition sequence of an extension probe is positioned 3′ to any detection sequence (e.g., barcode sequence) in the extension probe. In some embodiments, the target recognition sequence comprises a sequence that is substantially complementary to a portion of a target nucleic acid in the reporter oligonucleotide. In some embodiments, the target recognition sequence and the target sequence in the reporter oligonucleotide are at least 50%, at least 60%, at least 70%, at least 75%, at least 80%, at least 85%, at least 90%, at least 92%, at least 94%, at least 95%, at least 96%, at least 97%, at least 98%, at least 99%, or 100% complementary.

In some aspects, the extension probe comprises a stem loop structure. For example, the extension probe comprises a stem sequence, a loop sequence, and a complement of the stem sequence to form the stem loop structure. In some cases, the extension probe comprises one or more additional sequences outside of the stem and loop sequences. In some embodiments, the extension probe comprises a loop domain between the detection sequence and a complement of the detection sequence. In some embodiments, the extension probe comprises a loop domain comprising the detection sequence. In some embodiments, the extension probe comprises from 5′ to 3′: the complement of the detection sequence-the loop domain-the detection sequence-the target recognition sequence. In some embodiments, the extension probe is a linear oligonucleotide comprising from 5′ to 3′: the detection sequence-the target recognition sequence.

Attachment (coupling) of the reporter oligonucleotides or an extension product thereof to the labeling agents may be achieved through any of a variety of direct or indirect, covalent or non-covalent associations or attachments. For example, oligonucleotides extension products thereof may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Protein (e.g., antibody) and oligonucleotide biotinylation techniques are available. As a non-limiting example, in some cases click reaction chemistry is used to couple reporter oligonucleotides to labeling agents. Commercially available kits, such as those from Thunder-Link® and Abcam, and techniques common in the art may be used to couple reporter oligonucleotides or extension products thereof to labeling agents as appropriate. In another example, a labeling agent or an extension product thereof is indirectly (e.g., via hybridization) coupled to a reporter oligonucleotide comprising a barcode sequence that identifies the label agent. For instance, the labeling agent or an extension product thereof may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some embodiments, the reporter oligonucleotides or extension products thereof are releasable from the labeling agent, such as upon application of a stimulus. For example, the reporter oligonucleotide or extension product thereof may be attached to the labeling agent through a labile bond (e.g., chemically labile, photolabile, thermally labile, etc.) as generally described for releasing molecules from supports elsewhere herein.

As illustrated in FIG. 4A-4C, in some embodiments, the method comprises hybridizing a reporter oligonucleotide to an extension probe. Although an extension probe comprising a stem loop structure (e.g., a hairpin) is illustrated in FIG. 4A-4C, the extension probe can be a linear nucleic acid molecule that can be used to provide a template for the extension by the polymerase. The extension may hybridize to a reporter oligonucleotide prior to attachment to the labeling agent or to a reporter oligonucleotide already attached to a labeling agent. After incubating the sample to allow the extension probe to hybridize to the target reporter oligonucleotide, extension of a 3′ end of the target reporter oligonucleotide is performed using a sequence of the extension probe as template. In some embodiments, the generated concatemer is detected using methods described herein. For example, the generated concatemer is detected using sequential hybridization cycles of detectably labeled probes that directly or indirectly hybridize to detection sequences or amplification sequences or complements thereof (e.g., barcode sequences) or subunits thereof in the extension products (e.g., in the generated extended concatemer). In some embodiments, a sequence corresponding to the reporter oligonucleotide is detected.

Provided herein is a method comprising: hybridizing an extension probe to a reporter oligonucleotide, wherein the extension probe comprises (i) a target recognition sequence complementary to a target sequence of the reporter oligonucleotide, and (ii) a detection sequence; extending the reporter oligonucleotide with a polymerase using the extension probe as a template, thereby generating an extended nucleic acid molecule; and performing further extension of the extended nucleic acid molecule using a plurality of additional extension probes, wherein an additional extension probe of the plurality of additional extension probe comprises (i) at least a portion of the detection sequence of the extension probe or a complement thereof, and (ii) an amplification sequence; thereby generating an extended concatemer comprising the reporter oligonucleotide, the detection sequence or a complement thereof and the amplification sequence or a complement thereof.

Provided herein is a method comprising: hybridizing an extension probe to a reporter oligonucleotide, wherein the extension probe comprises (i) a target recognition sequence complementary to a target sequence of the reporter oligonucleotide, and (ii) a detection sequence; extending the reporter oligonucleotide with a polymerase using the extension probe as a template, thereby generating an extended nucleic acid molecule; performing further extension of the extended nucleic acid molecule using a plurality of additional extension probes, wherein an additional extension probe of the plurality of additional extension probe comprises (i) at least a portion of the detection sequence of the extension probe or a complement thereof, and (ii) an amplification sequence, thereby generating an extended concatemer comprising the reporter oligonucleotide, the detection sequence or a complement thereof and the amplification sequence or a complement thereof; and attaching the extended concatemer to a labeling agent. In some embodiments, the method of generating a labeling agent attached to the extended concatemer is performed in vitro. In some embodiments, a labeling agent comprising an extended concatemer is contacted with a biological sample (e.g., a cell or tissue sample).

Provided herein is a method comprising: hybridizing an extension probe to a reporter oligonucleotide attached to a labeling agent, wherein the extension probe comprises (i) a target recognition sequence complementary to a target sequence of the reporter oligonucleotide, and (ii) a detection sequence; extending the reporter oligonucleotide with a polymerase using the extension probe as a template, thereby generating an extended nucleic acid molecule; and performing further extension of the extended nucleic acid molecule using a plurality of additional extension probes, wherein an additional extension probe of the plurality of additional extension probe comprises (i) at least a portion of the detection sequence of the extension probe or a complement thereof, and (ii) an amplification sequence, thereby generating an extended concatemer comprising the reporter oligonucleotide, the detection sequence or a complement thereof and the amplification sequence or a complement thereof. In some embodiments, the method of generating a labeling agent attached to the extended concatemer is performed in vitro. In some embodiments, a labeling agent comprising an extended concatemer is contacted with a biological sample (e.g., a cell or tissue sample).

Provided herein is a method comprising: hybridizing an extension probe to a reporter oligonucleotide attached to a labeling agent, wherein the extension probe comprises (i) a target recognition sequence complementary to a target sequence of the reporter oligonucleotide, and (ii) a detection sequence; extending the reporter oligonucleotide with a polymerase using the extension probe as a template, thereby generating an extended nucleic acid molecule; and performing further extension of the extended nucleic acid molecule using a plurality of additional extension probes, wherein an additional extension probe of the plurality of additional extension probe comprises (i) at least a portion of the detection sequence of the extension probe or a complement thereof, and (ii) an amplification sequence, thereby generating an extended concatemer comprising the reporter oligonucleotide, the detection sequence or a complement thereof and the amplification sequence or a complement thereof. In some embodiments, the method of generating a labeling agent attached to the extended concatemer is performed in situ. In some embodiments, the extension and further extension is performed in a biological sample (e.g., a cell or tissue sample). In some embodiments, the extension and further extension is performed after the labeling agent binds to an analyte in a biological sample (e.g., a cell or tissue sample).

In some embodiments, the plurality of additional extension probes comprises a first additional extension probe and a second extension probe, wherein the first additional extension probe and the second extension probe comprises different amplification sequences. In some embodiments, the method further comprises detecting the detection sequence and/or a complement thereof. In some embodiments, the method further comprises detecting the amplification sequence and/or a complement thereof. In some embodiments, the detection sequence comprises a barcode sequence that identifies the labeling agent. In some embodiments, the detection sequence comprises a barcode sequence that identifies the reporter oligonucleotide corresponding to the labeling agent. In some embodiments, the detection sequence comprises a barcode sequence that identifies the analyte (e.g., polypeptide) bound by the labeling agent. In some embodiments, the amplification sequence comprises a barcode sequence that identifies the labeling agent. In some embodiments, the amplification sequence comprises a barcode sequence that identifies the reporter oligonucleotide corresponding to the labeling agent. In some embodiments, the amplification sequence comprises a barcode sequence that identifies the analyte (e.g., polypeptide) bound by the labeling agent. In some embodiments, the barcode sequence comprises two or more barcode subunits. In some embodiments, the extended nucleic acid molecule comprises the detection sequence or a complement thereof and at least two copies of the amplification sequence or a complement thereof.

In some embodiments, the extension probe comprises a stem loop structure. In some embodiments, the additional extension probe comprises a stem loop structure. In some embodiments, the extension probe comprises a stopper molecule or a stopper modification. In some embodiments, the additional extension probe comprises a stopper molecule or a stopper modification. In some embodiments, the stopper molecule or the stopper modification prevents extension of the extended nucleic acid molecule. In some embodiments, the stopper molecule or the stopper modification is located in the loop structure of the extension probe. In some embodiments, the stopper molecule or the stopper modification is located in the loop structure of the additional extension probe. In some embodiments, the stopper molecule or the stopper modification is located at the end of a stem structure of the extension probe. In some embodiments, the stopper molecule or the stopper modification is located at the end of a stem structure of the additional extension probe. In some embodiments, the stopper molecule or stopper modification comprises a chemical modification. In some embodiments, the chemical modification is a phosphoramidite.

In some embodiments, the method comprises imaging the extended nucleic acid molecule comprising the reporter oligonucleotide. In some embodiments, the method comprises imaging the extended concatemer comprising the reporter oligonucleotide. In some embodiments, the imaging comprises detecting a signal associated with a detectably labeled probe that directly or indirectly binds to the extended nucleic acid molecule and/or the extended concatemer comprising the reporter oligonucleotide. In some embodiments, the detectably labeled probe is a fluorescently labeled probe.

In some embodiments, the sequence of the extended nucleic acid molecule or the extended concatemer is analyzed by sequential hybridization, sequencing by hybridization, sequencing by ligation, sequencing-by-synthesis (SBS), sequencing-by-avidity (SBA), sequencing-by-binding (SBB), or a combination thereof. In some embodiments, the sequence of the extended nucleic acid molecule or extended concatemer is analyzed by single nucleotide sequencing by synthesis.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample. In some embodiments, performing the extension of the target RNA or an extension thereof and performing extension of the reporter oligonucleotides is in the same reaction mixture. In some embodiments, performing the extension of the target RNA or an extension thereof and performing extension of the reporter oligonucleotides is performed in two distinct mixtures. In some embodiments, performing the extension of the target RNA or an extension thereof and performing extension of the reporter oligonucleotides uses the same polymerase. In some embodiments, performing the extension of the target RNA or an extension thereof and performing extension of the reporter oligonucleotides uses different polymerase.

In some embodiments, the extension of a plurality of different reporter oligonucleotides occurs in the same reaction mixture. In some embodiments, the extension of a plurality of different reporter oligonucleotides is performed using a plurality of different extension probes.

In some embodiments, the method comprises generating a signal code sequence at one or more locations in a sample, the signal code sequence comprising signal codes corresponding to the signals (or absence thereof) associated with detectable probes for in situ hybridization that are sequentially applied to the sample, wherein the signal code sequence corresponds to an analyte in the sample, thereby detecting the analyte at the one or more of the multiple locations in the sample.

In some embodiments, the method comprises imaging the biological sample to detect the generated extension product, e.g., an extended nucleic acid molecule or an extended concatemer comprising the target RNA or a portion thereof. In some embodiments, the imaging comprises detecting a signal associated with a fluorescently labeled probe that directly or indirectly binds to the generated extension product. In some instances, the fluorescently labeled probe directly binds to the generated extension product, e.g., an extended nucleic acid molecule or an extended concatemer comprising the target RNA or a portion thereof. In some embodiments, the fluorescently labeled probe indirectly binds to the generated extension product, e.g., an extended nucleic acid molecule or an extended concatemer comprising the target RNA or a portion thereof. In some embodiments, one or more intermediate probes binds to a barcode sequence or subunit thereof in the generated extension product, and the one or more intermediate probes comprise one or more barcode sequences corresponding to one or more fluorescently labeled probes. In some embodiments, the fluorescently labeled probe hybridizes to a corresponding barcode sequence in the intermediate probe.

In some embodiments, a sequence of the generated extension product, e.g., an extended nucleic acid molecule or an extended concatemer comprising the target RNA or a portion thereof is analyzed at a location in the biological sample or a matrix embedding the biological sample. In some embodiments, the sequence of the generated extension product, e.g., an extended nucleic acid molecule or an extended concatemer comprising the target RNA or a portion thereof is analyzed by sequential hybridization, sequencing by hybridization, sequencing by ligation, sequencing-by-synthesis (SBS), sequencing-by-avidity (SBA), sequencing-by-binding (SBB), or a combination thereof. In some embodiments, the sequence of the extension product comprises one or more barcode sequences or complements thereof (e.g., one or more barcode sequences or complements thereof that individually or in combination identify the target RNA or reporter oligonucleotide corresponding to a non-nucleic acid analyte).

In some embodiments, a target RNA described herein is associated with one or more barcode(s) present in an extension probe or the additional extension probes. In some embodiments, an extension probe or additional extension probe comprises at least two, three, four, five, six, seven, eight, nine, ten, or more barcodes. Barcodes can spatially-resolve molecular components found in biological samples, for example, within a cell or a tissue sample. A barcode can be attached to an analyte or to another moiety or structure in a reversible or irreversible manner. In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, the barcode sequence comprises one or more barcode positions each comprising one or more barcode subunits. In some embodiments, a barcode position in the barcode sequence partially overlaps an adjacent barcode position in the barcode sequence. In some embodiments, the first detectable probe and the subsequent detectable probe are in a set of detectable probes each comprising the same recognition sequence and a reporter. In some embodiments, the reporter of each detectable probe in the set comprises a binding site for a reporter probe comprising a detectable moiety. In some embodiments, the reporter probe binding site of the first detectable probe and the reporter probe binding site of the subsequent detectable probe are the same. In some embodiments, the reporter probe binding site of the first detectable probe and the reporter probe binding site of the subsequent detectable probe are different. In some embodiments, the detectable moiety is a fluorophore and the signal code sequence is a fluorophore sequence uniquely assigned to the target nucleic acid (e.g., target RNA). In some embodiments, the detectable probes in the set are contacted with the sample sequentially in a pre-determined sequence which corresponds to the signal code sequence assigned to the barcode sequence. In some embodiments, the detectable probes in the set are contacted with the sample to determine signal codes in the signal code sequence until sufficient signal codes have been determined to decode the barcode sequence, thereby identifying the target nucleic acid (e.g., target RNA or reporter oligonucleotide corresponding to a non-nucleic acid analyte).

In some aspects, the provided methods involve analyzing, e.g., detecting or determining, one or more sequences present in generated extension product, e.g., an extended nucleic acid molecule or an extended concatemer comprising the target RNA or a portion thereof or reporter oligonucleotide corresponding to a non-nucleic acid analyte. In some cases, the analysis is performed on one or more images captured, and may comprise processing the image(s) and/or quantifying signals observed. For example, the analysis may comprise processing information of one or more cell types, one or more types of biomarkers, a number or level of a biomarker, and/or a number or level of cells detected in a particular region of the sample. In some embodiments, the analysis comprises detecting a sequence e.g., a barcode present in the sample. In some embodiments, the analysis includes quantification of puncta (e.g., if amplification products are detected). In some cases, the analysis includes determining whether particular cells and/or signals are present that correlate with one or more biomarkers from a particular panel. In some embodiments, the obtained information may be compared to a positive and negative control, or to a threshold of a feature to determine if the sample exhibits a certain feature or phenotype. In some cases, the information may comprise signals from a cell, a region, and/or comprise readouts from multiple detectable labels. In some case, the analysis further includes displaying the information from the analysis or detection. In some embodiments, software may be used to automate the processing, analysis, and/or display of data.

In any of the embodiments herein, a sequence of the generated extension product, e.g., an extended nucleic acid molecule or an extended concatemer comprising the target RNA or a portion thereof, comprises one or more barcode sequences or complements thereof. In any of the embodiments herein, the sequence of the generated extension product, e.g., an extended nucleic acid molecule or an extended concatemer comprising the target RNA or a portion thereof comprises one or more barcode sequences or complements thereof. In any of the embodiments herein, the sequence of the generated extension product, e.g., an extended nucleic acid molecule or an extended concatemer comprising the reporter oligonucleotide corresponding to a non-nucleic acid analyte, comprises one or more barcode sequences or complements thereof.

In some aspects, the detecting comprises contacting the biological sample with one or more detectably labeled probes that directly or indirectly hybridize to the extension product, and dehybridizing the one or more detectably labeled probes from the extension product. In any of the embodiments herein, the contacting and dehybridizing can be repeated with the one or more detectably labeled probes and/or one or more other detectably labeled probes that directly or indirectly hybridize to the extension product.

In some aspects, the detecting comprises contacting the biological sample with one or more intermediate probes that directly or indirectly hybridize to the extension product, wherein the one or more intermediate probes are detectable using one or more detectably labeled probes. In any of the embodiments herein, the detecting further comprises dehybridizing the one or more intermediate probes and/or the one or more detectably labeled probes from the extension product. In any of the embodiments herein, the contacting and dehybridizing are repeated with the one or more intermediate probes, the one or more detectably labeled probes, one or more other intermediate probes, and/or one or more other detectably labeled probes.

In some embodiments, the analysis and/or sequence determination comprises detecting all or a portion of the generated extension product, e.g., an extended nucleic acid molecule or an extended concatemer comprising the target RNA or a portion thereof and/or in situ hybridization to the generated extension products. In some embodiments, the sequencing involves sequencing by hybridization, sequencing by ligation, and/or fluorescent in situ sequencing, hybridization-based in situ sequencing and/or wherein the in situ hybridization comprises sequential fluorescent in situ hybridization. In some embodiments, the detection or determination comprises hybridizing to the extension product a detection oligonucleotide labeled with a fluorophore, an isotope, a mass tag, or a combination thereof. In some embodiments, the detection or determination comprises imaging the generated extension product, e.g., an extended nucleic acid molecule or an extended concatemer comprising the target RNA or a portion thereof. In some embodiments, the target nucleic acid is an mRNA in a tissue sample, and the detection or determination is performed when the target nucleic acid and/or the generated extension product is in situ in the tissue sample. In some embodiments, the generated extension product, e.g., an extended nucleic acid molecule or an extended concatemer, comprises (e.g., is covalently attached to) the cleaved target at a location in the biological sample. In some embodiments, the analytes (e.g., target RNAs), probes and/or extension products described herein are anchored to a polymer. For example, the polymer matrix can be a hydrogel. In some embodiments, cross-linking of the matrix or components to be anchored to the matrix can be performed chemically and/or photochemically, or alternatively by any other suitable hydrogel-formation method.

In some aspects, the provided methods comprise imaging the generated extension product, e.g., an extended nucleic acid molecule or an extended concatemer comprising the target RNA or a portion thereof, for example, via binding of the detectably labeled probe detecting the detectable label. In some embodiments, the detectably labeled probe comprises a detectable label that can be measured and quantitated. The detectable label can be any label that can be measured, e.g., fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like. In some embodiments, a detectable probe containing a detectable label can be used to detect one or more extension products according to the methods described herein. In some embodiments, the methods involve incubating the detectable probe containing the detectable label with the sample, washing unbound detectable probe, and detecting the label, e.g., by imaging.

In some embodiments, the detectable label is a fluorophore that comprises a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, cyanine-3 (Cy3), cyanine-5 (Cy5), cyanine-7 (Cy7), rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. Background fluorescence can arise from a variety of sources, including aldehyde fixation, extracellular matrix components, red blood cells, lipofuscin, and the like. Tissue background fluorescence (or autofluorescence) can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (Max Vision Biosciences), and/or a very intense black dye (e.g., Sudan Black, or comparable dark chromophore).

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes), all of which are herein incorporated by reference in their entireties. Examples of detectable nanoparticles e.g., quantum dots, comprise those described in, for example, U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264, all of which are herein incorporated by reference in their entireties. As used herein, the term “fluorescent label” comprises a signaling moiety that conveys information through the fluorescent absorption and/or emission properties of one or more molecules. Examples of fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3™-dCTP (cyanine 3-dCTP), Cy3™-dUTP (cyanine 3-dUTP), Cy5™-dCTP (cyanine 5-dCTP), Cy5™-dUTP (cyanine 5 dUTP) (Amersham Biosciences, Piscataway, N.J.), fluorescein-12-dUTP, tetramethylrhodamine-6-dUTP, TEXAS RED®-5-dUTP (red fluorescent dye-dUTP), CASCADE® BLUE-7-dUTP (blue fluorescent dye-dUTP), BODIPY™ FL-14-dUTP (green fluorescent dye-dUTP), BODIPY™ TMR-14-dUTP (orange fluorescent dye-dUTP), BODIPY™ TR-14-dUTP (red fluorescent dye-dUTP), RHODAMINE GREEN™-5-dUTP (green fluorescent dye-dUTP), OREGON GREEN™ 488-5-dUTP (green fluorescent dye-dUTP), TEXAS RED™-12-dUTP (red fluorescent dye-dUTP), BODIPY™ 630/650-14-dUTP (far red fluorescent dye-dUTP), BODIPY™ 650/665-14-dUTP (far red fluorescent dye-dUTP), ALEXA FLUOR™ 488-5-dUTP (green fluorescent dye-dUTP), ALEXA FLUOR™ 532-5-dUTP (yellow fluorescent dye-dUTP), ALEXA FLUOR™ 568-5-dUTP (red/orange fluorescent dye-dUTP), ALEXA FLUOR™ 594-5-dUTP (red fluorescent dye-dUTP), ALEXA FLUOR™ 546-14-dUTP (orange fluorescent dye-dUTP), fluorescein-12-UTP, tetramethylrhodamine-6-UTP, TEXAS RED™-5-UTP (red fluorescent dye-UTP), mCherry, CASCADE® BLUE-7-UTP (blue fluorescent dye-UTP), BODIPY™ FL-14-UTP (green fluorescent protein-UTP), BODIPY™ TMR-14-UTP (orange fluorescent dye-UTP), BODIPY™ TR-14-UTP (red fluorescent dye-UTP), RHODAMINE GREEN™-5-UTP (green fluorescent dye-UTP), ALEXA FLUOR™ 488-5-UTP (green fluorescent dye-UTP), and ALEXA FLUOR™ 546-14-UTP (orange fluorescent dye-UTP) (Molecular Probes, Inc. Eugene, Oreg.). Methods are known for custom synthesis of nucleotides having other fluorophores.

Other fluorophores available for post-synthetic attachment comprise, but are not limited to, ALEXA FLUOR™ dyes (fluorescent dyes) such as ALEXA FLUOR™ 350 (blue fluorescent dye), ALEXA FLUOR™ 594 (red fluorescent dye), and ALEXA FLUOR™ 647 (far red fluorescent dye); BODIPY™ dyes (fluorescent dyes) such as BODIPY™ FL (green fluorescent dye), BODIPY™ TMR (orange fluorescent dye), and BODIPY™ 650/665 (far red fluorescent dye); Cascade® Blue (blue fluorescent dye), Cascade® Yellow (yellow fluorescent dye), Dansyl, lissamine rhodamine B, Marina Blue™ (blue fluorescent dye), Oregon Green™ 488, Oregon Green™ 514, Pacific Blue, rhodamine 6G, rhodamine green, rhodamine red, tetramethyl rhodamine, Texas Red® (red fluorescent dye) (available from Molecular Probes, Inc., Eugene, Oreg.), Cy2™ (cyanine 2), Cy3.5™ (cyanine 3.5), Cy5.5™ (cyanine 5.5), and Cy7™ (cyanine 7) (Amersham Biosciences, Piscataway, N.J.). FRET tandem fluorophores may also be used, comprising, but not limited to, PerCP-Cy™5.5 (far red fluorescent tandem fluorophore), PE-Cy™5 (red fluorescent tandem fluorophore), PE-Cy™5.5 (red fluorescent tandem fluorophore), PE-Cy™7 (far red fluorescent tandem fluorophore), PE-Texas Red® (red fluorescent tandem fluorophore), APC-Cy™7 (far red fluorescent tandem fluorophore), PE-Alexa™ dyes (e.g., 610, 647, 680), and APC-Alexa™ dyes.

In some cases, metallic silver or gold particles may be used to enhance signal from fluorescently labeled nucleotide and/or polynucleotide sequences (Lakowicz et al. (2003) Bio Techniques 34:62).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection. In any of the embodiments herein, an antibody can be an antibody molecule of any class, or any sub-fragment thereof, such as a Fab.

Other suitable labels for a polynucleotide sequence may comprise fluorescein (FAM), digoxigenin, dinitrophenol (DNP), dansyl, biotin, bromodeoxyuridine (BrdU), hexahistidine (6×His (SEQ ID NO: 31)), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or a polynucleotide sequence can be indirectly labeled, especially with a hapten that is then bound by a capture agent, e.g., as disclosed in U.S. Pat. Nos. 5,344,757, 5,702,888, and 5,198,537, all of which are herein incorporated by reference in their entireties. Many different hapten-capture agent pairs are available for use. Example haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detection probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a simpler set up like an epifluorescence microscope, or a more complicated design such as a confocal microscope, which uses optical sectioning to get better resolution of the fluorescent image.

In some embodiments, confocal microscopy is used for detection and imaging of the detection probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity, so long exposures can be required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large, clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, sequencing or sequence detection is performed in situ. In situ sequencing typically involves incorporation of a labeled nucleotide (e.g., fluorescently labeled mononucleotides or dinucleotides) in a sequential, template-dependent manner or hybridization of a labeled primer to a nucleic acid template such that the identities (i.e., nucleotide sequence) of the incorporated nucleotides or labeled primer extension products can be determined, and consequently, the nucleotide sequence of the corresponding template nucleic acid.

In some embodiments, analyzing, e.g., detecting or determining, one or more sequences present in the extension product is performed using a base-by-base sequencing method, e.g., sequencing-by-synthesis (SBS), sequencing-by-avidity (SBA) or sequencing-by-binding (SBB). In some embodiments, the biological sample is contacted with a sequencing primer and base-by-base sequencing using a cyclic series of nucleotide incorporation or binding, respectively, thereby generating extension products of the sequencing primer is performed followed by removing, cleaving, or blocking the extension products of the sequencing primer.

Generally in sequencing-by-synthesis methods, a first population of detectably labeled nucleotides (e.g., dNTPs) are introduced to contact a template nucleotide (e.g., a barcode sequence in the RCP) hybridized to a sequencing primer, and a first detectably labeled nucleotide (e.g., A, T, C, or G nucleotide) is incorporated by a polymerase to extend the sequencing primer in the 5′ to 3′ direction using a complementary nucleotide (a first nucleotide residue) in the template nucleotide as template. A signal from the first detectably labeled nucleotide can then be detected. The first population of nucleotides may be continuously introduced, but in order for a second detectably labeled nucleotide to incorporate into the extended sequencing primer, nucleotides in the first population of nucleotides that have not incorporated into a sequencing primer are generally removed (e.g., by washing), and a second population of detectably labeled nucleotides are introduced into the reaction. Then, a second detectably labeled nucleotide (e.g., A, T, C, or G nucleotide) is incorporated by the same or a different polymerase to extend the already extended sequencing primer in the 5′ to 3′ direction using a complementary nucleotide (a second nucleotide residue) in the template nucleotide as template. Thus, in some embodiments, cycles of introducing and removing detectably labeled nucleotides are performed.

In some embodiments, the base-by-base sequencing comprises using a polymerase that is fluorescently labeled. In some embodiments, the base-by-base sequencing comprises using a polymerase-nucleotide conjugate comprising a fluorescently labeled polymerase linked to a nucleotide moiety that is not fluorescently labeled. In some embodiments, the base-by-base sequencing comprises using a multivalent polymer-nucleotide conjugate comprising a polymer core, multiple nucleotide moieties, and one or more fluorescent labels.

In some embodiments, sequencing can be performed by sequencing-by-synthesis (SBS). In some embodiments, a sequencing primer is complementary to sequences at or near the one or more detection sequence or amplification sequences (e.g., barcode(s)). In such embodiments, sequencing-by-synthesis can comprise reverse transcription and/or amplification in order to generate a template sequence from which a primer sequence can bind. In some embodiments, the SBS methods comprise incorporation and/or imaging such as those described in US 2013/0079232; use reagents including, for example, modified and/or labelled nucleotides such as those described in US 2007/0166705 and U.S. Pat. No. 7,057,026; polymerases such as those described in US 2006/0281109, all of which are herein incorporated by reference in their entireties.

In some embodiments, sequencing is performed by sequencing-by-binding (SBB). Various aspects of SBB are described in U.S. Pat. No. 10,655,176 B2, the content of which is herein incorporated by reference in its entirety. In some embodiments, SBB comprises performing repetitive cycles of detecting a stabilized complex that forms at each position along the template nucleic acid to be sequenced (e.g. a ternary complex that includes the primed template nucleic acid, a polymerase, and a cognate nucleotide for the position), under conditions that prevent covalent incorporation of the cognate nucleotide into the primer, and then extending the primer to allow detection of the next position along the template nucleic acid. In the sequencing-by-binding approach, detection of the nucleotide at each position of the template occurs prior to extension of the primer to the next position. Generally, the methodology is used to distinguish the four different nucleotide types that can be present at positions along a nucleic acid template by uniquely labelling each type of ternary complex (i.e. different types of ternary complexes differing in the type of nucleotide it contains) or by separately delivering the reagents needed to form each type of ternary complex. In some instances, the labeling may comprise fluorescence labeling of, e.g., the cognate nucleotide or the polymerase that participate in the ternary complex.

In some embodiments, sequencing is performed by sequencing-by-avidity (SBA). Some aspects of SBA approaches are described in U.S. Pat. No. 10,768,173 B2, the content of which is herein incorporated by reference in its entirety. In some embodiments, SBA comprises detecting a multivalent binding complex formed between a fluorescently-labeled polymer-nucleotide conjugate, and a one or more primed target nucleic acid sequences (e.g., barcode sequences). Fluorescence imaging is used to detect the bound complex and thereby determine the identity of the N+1 nucleotide in the target nucleic acid sequence (where the primer extension strand is N nucleotides in length). Following the imaging, the multivalent binding complex is disrupted and washed away, the correct blocked nucleotide is incorporated into the primer extension strand, and the sequencing cycle is repeated.

In some embodiments, sequencing is performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in U.S. Pat. No. 5,599,675 the content of which is herein incorporated by reference in its entirety.

In some embodiments, nucleic acid hybridization is used for sequencing. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004), all of which are herein incorporated by reference in their entireties. In some embodiments, detection of the barcode sequences is performed by sequential hybridization of probes to the barcode sequences or complements thereof and detecting complexes formed by the probes and barcode sequences or complements thereof. In some cases, each barcode sequence or complement thereof is assigned a sequence of signal codes that identifies the barcode sequence or complement thereof (e.g., a temporal signal signature or code that identifies the analyte), and detecting the barcode sequences or complements thereof can comprise decoding the barcode sequences of complements thereof by detecting the corresponding sequences of signal codes detected from sequential hybridization, detection, and removal of sequential pools of intermediate probes and the universal pool of detectably labeled probes. In some cases, the sequences of signal codes comprise fluorophore sequences assigned to the corresponding barcode sequences or complements thereof. In some embodiments, the detectably labeled probes are fluorescently labeled. In some embodiments, the barcode sequence or complement thereof is performed by sequential probe hybridization as described in US 2021/0340618, the content of which is herein incorporated by reference in its entirety.

In some embodiments, real-time monitoring of DNA polymerase activity can be used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181, all of which are herein incorporated by reference in their entireties.

In some aspects, the analysis and/or sequence determination can be carried out at room temperature for best preservation of tissue morphology with low background noise and error reduction. In some embodiments, the analysis and/or sequence determination comprises eliminating error accumulation as sequencing proceeds.

In some embodiments, the analysis and/or sequence determination involves washing to remove unbound polynucleotides, thereafter revealing a fluorescent product for imaging.

### III. Compositions, Systems and Kits

In some aspects, provided herein are compositions, systems or kits comprising an extension probe comprising i) a target recognition sequence complementary to a target sequence at the 3′ end of a target RNA, and ii) a detection sequence and a polymerase for performing extension of a target RNA using the extension probe as template. In some embodiments, the compositions, systems or kits comprise a plurality of extension probes and a plurality of additional extension probes (e.g., as described in Section II.A). In some embodiments, the compositions, systems or kits comprise reagents for cleaving the target RNA (e.g., as described in Section II.B). In some embodiments, the compositions, systems or kits comprise nucleic acid oligonucleotides and/or RNase H for cleaving target RNA. In some embodiments, the compositions, systems or kits comprise RNA-cutting enzyme (e.g., Argonaute protein or CRISPR effector protein for cleaving target RNA. In some embodiments, the compositions, systems or kits comprise reagents for sequencing, or detectably labeled probes, and/or intermediate probes described herein for detecting a sequence of the extension product generated using the extension probe (e.g., as described in Section II.D). Also provided herein are compositions, systems or kits for analyzing an analyte in a biological sample according to any of the methods described herein. In some embodiments, provided herein is a kit comprising any of the nucleic acid oligonucleotides described herein (e.g., for duplex formation with target RNA and RNase H cleavage of the target RNA). In some embodiments, the kit comprises RNase H. In some embodiments, the kit comprises a polymerase for extending a target RNA or an extension thereof. In some aspects, provided herein are compositions, systems or kits for extending a reporter oligonucleotide comprising: a polymerase, an extension probe, a labeling agent, a reporter oligonucleotide, and one or more additional extension probes. In some aspect, the system or kit further comprises reagents for attaching the reporter oligonucleotide to the labeling agent The various components of the kit may be present in separate containers or certain compatible components may be pre-combined into a single container. In some embodiments, the kits further contain instructions for using the components of the kit to practice the provided methods.

In some embodiments, the compositions, systems or kits comprise a plurality of extension probes and/or additional extension probes. In some embodiments, the compositions, systems or kits comprise a corresponding plurality of nucleic acid oligonucleotides for cleaving and analyzing a plurality of target RNAs. In some embodiments, the plurality of nucleic acid oligonucleotides comprises at least 2, at least 5, at least 10, at least 25, at least 50, at least 75, at least 100, at least 300, at least 1,000, at least 3,000, at least 10,000, at least 30,000, at least 50,000, at least 100,000, at least 250,000, at least 500,000, or at least 1,000,000 distinguishable nucleic acid oligonucleotides. In some embodiments, the kit comprises at least 2, at least 5, at least 10, at least 25, at least 50, at least 75, at least 100, at least 300, at least 1,000, at least 3,000, at least 10,000, at least 30,000, at least 50,000, at least 100,000, at least 250,000, at least 500,000, or at least 1,000,000 extension probes.

In some aspects, provided are compositions, systems or kits for analyzing a biological sample, comprising: a nucleic acid oligonucleotide, wherein the nucleic acid oligonucleotide is complementary to an oligonucleotide hybridization region in a target ribonucleic acid (RNA); b) an extension probe, wherein the extension probe comprises i) a target recognition sequence complementary to a target sequence at the 3′ end of the target RNA, and ii) a detection sequence; c) an RNase H for cleaving the oligonucleotide hybridization region of the target RNA when hybridized to the nucleic acid oligonucleotide; and d) a polymerase for performing extension of the cleaved target RNA using the detection sequence of the extension probe as template. In some embodiments, the compositions, systems, or kits further comprise reagents for an extension reaction including a mixture of dNTPs. In some aspects, the compositions, systems or kits further comprise an extension probe comprising a detection sequence and a target recognition sequence complementary to a reporter oligonucleotide corresponding to a labeling agent configured to bind to a non-nucleic acid analyte (e.g., a polypeptide).

In some aspects, provided herein is a kit or system for analyzing a biological sample, comprising: a nucleic acid oligonucleotide, wherein the nucleic acid oligonucleotide is complementary to an oligonucleotide hybridization region in a target ribonucleic acid (RNA); an extension probe, wherein the extension probe comprises i) a target recognition sequence complementary to a target sequence at the 3′ end of the target RNA, and ii) a detection sequence; and an RNase H for cleaving the oligonucleotide hybridization region of the target RNA when hybridized to the nucleic acid oligonucleotide.

In some aspects, provided herein is system comprising a solid support having a biological sample attached thereto. In some instances, the biological sample is a cell sample or a tissue sample. In some aspects, provided herein is system comprising an optical detection system configured to detect the detection sequence or a complement thereof in the extended concatemer.

In some instances, the compositions, systems or kits further comprises reagents for performing sequencing by ligation, sequencing-by-synthesis (SBS), sequencing-by-avidity (SBA), sequencing-by-binding (SBB), or a combination thereof. In some instances, the kit or system further comprises one or more intermediate probes and a universal pool of detectably labeled probes (e.g., as described in Section II).

In some embodiments, the compositions, systems or kits contain reagents and/or consumables required for performing one or more operations of the provided methods. In some embodiments, the kit or system contains reagents for fixing, embedding, and/or permeabilizing the biological sample. In some embodiments, the kits contain reagents, such as enzymes and buffers for extension, such as polymerases. In some aspects, the kit can also comprise any of the reagents described herein, e.g., wash buffers. In some embodiments, the compositions, systems or kits contain reagents for detection and/or sequencing, such as detectably labeled probes for binding to one or more barcode sequences or complements thereof, or detectable labels.

### IV. Biological Sample Preparation

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or lipids. The biological sample can be obtained as a tissue sample, such as a tissue section, biopsy, a core biopsy, a cell pellet, a cell block, a needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Biological samples can be derived from a homogeneous culture or population of the subjects or organisms mentioned herein or alternatively from a collection of several different organisms. Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent operations in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose. In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of operations can be performed to prepare or process a biological sample for and/or during an assay. Except where indicated otherwise, the preparative or processing operations described below can generally be combined in any manner and in any order to appropriately prepare or process a particular sample for and/or analysis.

### (i) Preparation

In some embodiments, a biological sample is harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick. More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analyzed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

In some embodiments, the biological sample (e.g., a tissue section as described above) is prepared by deep freezing at a temperature suitable to maintain or preserve the integrity (e.g., the physical characteristics) of the tissue structure. In some embodiments, the frozen tissue sample is sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than-15° C., less than −20° C., or less than −25° C.

In some embodiments, the biological sample is prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes). In some embodiments, the biological sample (e.g., FFPE sample) is permeable after deparaffinization. In some embodiments, processing of the biological sample, such as de-waxing, allows the biological sample to become permeabilized.

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, the methods provided herein comprises one or more post-fixing (also referred to as postfixation) operations. In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked.

In some embodiments, a biological sample can be permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the content of which is herein incorporated by reference in its entirety. Any suitable method for sample permeabilization can generally be used in connection with the samples described herein.

In some embodiments, the biological sample can be permeabilized by any suitable methods. For example, one or more lysis reagents can be added to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes. Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise an operation for increasing accessibility of a nucleic acid for binding, e.g., a denaturation operation to open up DNA in a cell for hybridization by a probe. For example, proteinase K treatment may be used to free up DNA with proteins bound thereto.

### (ii) Embedding

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample. Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some aspects, a biological sample can be embedded in any of a variety of other embedding materials to provide structural substrate to the sample prior to sectioning and other handling operations. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

In some embodiments, the biological sample is reversibly cross-linked prior to or during an in situ assay. In some aspects, the analytes, polynucleotides and/or the generated extension product is crosslinked to a polymer matrix. For example, the polymer matrix can be a hydrogel. In some embodiments, one or more of the polynucleotide probe(s) and/or generated extension product can be modified to contain functional groups that can be used as an anchoring site to attach the polynucleotide probes and/or generated extension product to a polymer matrix. In some embodiments, a modified probe comprising oligo dT may be used to bind to mRNA molecules of interest, followed by reversible or irreversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. No. 6,391,937 and materials for sample expansion as described, for example, in U.S. Patent Application Publication Nos. 2017/0253918 and 2018/0052081, the entire contents of each of which are incorporated herein by reference.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample comprises cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347 (6221): 543-548, 2015, the entire content of which is incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible. In some embodiments, HTC reagents are added to the hydrogel before, contemporaneously with, and/or after polymerization. In some embodiments, a cell labeling agent is added to the hydrogel before, contemporaneously with, and/or after polymerization. In some embodiments, a cell-penetrating agent is added to the hydrogel before, contemporaneously with, and/or after polymerization.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and oligonucleotides. In some embodiments, optical labels are added to the hydrogel subunits before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a biological sample embedded in a matrix (e.g., a hydrogel) can be isometrically expanded. Isometric expansion methods that can be used include hydration, a preparative operation in expansion microscopy, as described in, e.g., Chen et al., Science 347 (6221): 543-548, 2015 and U.S. Pat. No. 10,059,990, all of which are herein incorporated by reference in their entireties. Isometric expansion of the sample can increase the spatial resolution of the subsequent analysis of the sample. The increased resolution in spatial profiling can be determined by comparison of an isometrically expanded sample with a sample that has not been isometrically expanded. In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(iii) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining operations may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Any suitable methods of destaining or discoloring a biological sample may be utilized and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65 (8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### V. Opto-Fluidic Instruments for Analysis of Biological Samples

Provided herein is an instrument having integrated optics and fluidics modules (an “opto-fluidic instrument” or “opto-fluidic system”) for preparing and/or detecting target molecules (e.g., nucleic acids, proteins, antibodies, etc.) in biological samples (e.g., one or more cells or a tissue sample) as described herein. In an opto-fluidic instrument, the fluidics module is configured to deliver one or more reagents (e.g., extension probes, additional extension probes, polymerases, reagents for cleaving the target RNAs, reagents for detecting sequences) to the biological sample and/or remove spent reagents therefrom. In some embodiments, the fluidics module is configured to deliver one or more probes (e.g., any as described in Section II). In some cases, the fluidics module is configured to remove the nucleic acid oligonucleotide(s) and/or RNA cleaving reagents (e.g., RNase H). In some cases, the fluidics module is configured to remove the probes and/or extension reagents. For example, one or more wash operations can be performed to remove the probes used for extension of the target RNA or portion thereof. In some embodiments, the fluidics module is configured to deliver and/or cycle one or more reagents (e.g., for extension to generate the extended concatemer). In some embodiments, the fluidics module is configured to deliver one or more detectably labeled probes and optionally intermediate probes to detect the generated extension products in the biological sample.

Additionally, the optics module is configured to illuminate the biological sample with light having one or more spectral emission curves (over a range of wavelengths) and subsequently capture one or more images of emitted light signals from the biological sample during one or more probing cycles (e.g., for detecting the generated extension products as described in Section II). In various embodiments, the captured images may be processed in real time and/or at a later time to determine the presence of the one or more target molecules in the biological sample, as well as three-dimensional position information associated with each detected target molecule. Additionally, the opto-fluidics instrument includes a sample module configured to receive (and, optionally, secure) one or more biological samples. In some instances, the sample module includes an X-Y stage configured to move the biological sample along an X-Y plane (e.g., perpendicular to an objective lens of the optics module).

In various embodiments, the opto-fluidic instrument is configured to analyze one or more target RNAs (e.g., as described in Section II) in their naturally occurring place (i.e., in situ) within the biological sample. In some embodiments, the opto-fluidic instrument is configured to process (e.g., perform library preparation) and analyze one or more target RNAs (e.g., as described in Section II) in relative spatial locations within the biological sample. For example, an opto-fluidic instrument may be an in-situ analysis system used to analyze a biological sample and detect target molecules including but not limited to DNA, RNA, proteins, antibodies, and/or the like. In some embodiments, the in situ analysis system is used to detect one or more extension products comprising target RNAs or a portion thereof according to the methods disclosed herein.

It is to be noted that, although the above discussion relates to an opto-fluidic instrument that can be used for in situ target molecule detection of extension products of target RNAs according to the methods disclosed herein, the discussion herein equally applies to any opto-fluidic instrument that employs any imaging or target molecule detection technique. That is, for example, an opto-fluidic instrument may include a fluidics module that includes fluids needed for establishing the experimental conditions required for the probing of target molecules in the sample. Further, such an opto-fluidic instrument may also include a sample module configured to receive the sample, and an optics module including an imaging system for illuminating (e.g., exciting one or more fluorescent probes within the sample) and/or imaging light signals received from the probed sample. The in-situ analysis system may also include other ancillary modules configured to facilitate the operation of the opto-fluidic instrument, such as, but not limited to, cooling systems, motion calibration systems, etc.

In various embodiments, the sample can be a biological sample (e.g., a tissue) that includes molecules such as DNA, RNA, proteins, antibodies, etc. For example, the sample can be a sectioned tissue that is treated to access the RNA thereof for hybridization of nucleic acid oligonucleotides such as extension probes (e.g., in Section II).

In various embodiments, the sample is placed in the opto-fluidic instrument for analysis and detection of the molecules (e.g., target RNAs and/or reporter oligonucleotides) in the sample. In various embodiments, the opto-fluidic instrument can be a system configured to facilitate the experimental conditions conducive for the detection of the target molecules. For example, the opto-fluidic instrument can include a fluidics module, an optics module, a sample module, and an ancillary module, and these modules may be operated by a system controller to create the experimental conditions for the detection of the molecules in the sample, as well as to facilitate the imaging of the probed sample (e.g., by an imaging system of the optics module). In various embodiments, the various modules of the opto-fluidic instrument may be separate components in communication with each other, or at least some of them may be integrated together.

In various embodiments, the sample module may be configured to receive the sample into the opto-fluidic instrument. For instance, the sample module may include a sample interface module (SIM) that is configured to receive a sample device (e.g., cassette) onto which the sample can be deposited. That is, the sample may be placed in the opto-fluidic instrument by depositing the sample (e.g., the sectioned tissue) on a sample device that is then inserted into the SIM of the sample module. In some instances, the sample module may also include an X-Y stage onto which the SIM is mounted. The X-Y stage may be configured to move the SIM mounted thereon (e.g., and as such the sample device containing the sample inserted therein) in perpendicular directions along the two-dimensional (2D) plane of the opto-fluidic instrument.

The experimental conditions that are conducive for the detection of the generated extension product, e.g., an extended nucleic acid molecule or an extended concatemer, in the sample may depend on the target molecule detection technique that is employed by the opto-fluidic instrument. For example, in various embodiments, the opto-fluidic instrument can be a system that is configured to detect extension products in the sample via hybridization of probes. In such cases, the experimental conditions can include molecule hybridization conditions that result in the intensity of hybridization of the extension product to a probe (e.g., detectably labeled probe) being significantly higher when the detectably labeled probe sequence is complementary to the extension product (e.g., to a barcode sequence or subunit in the extension product) than when there is a single-base mismatch. The hybridization conditions include the preparation of the sample using reagents such as washing/stripping reagents, hybridizing reagents, extension reagents etc., and such reagents may be provided by the fluidics module.

In various embodiments, the fluidics module may include one or more components that may be used for storing the reagents, as well as for transporting said reagents to and from the sample device containing the sample. For example, the fluidics module may include reservoirs configured to store the reagents, as well as a waste container configured for collecting the reagents (e.g., and other waste) after use by the opto-fluidic instrument to analyze and detect the molecules of the sample. Further, the fluidics module may also include pumps, tubes, pipettes, etc., that are configured to facilitate the transport of the reagent to the sample device (e.g., and as such the sample). For instance, the fluidics module may include pumps (“reagent pumps”) that are configured to pump washing/stripping reagents to the sample device for use in washing/stripping the sample (e.g., as well as other washing functions such as washing an objective lens of the imaging system of the optics module).

In various embodiments, the ancillary module can be a cooling system of the opto-fluidic instrument, and the cooling system may include a network of coolant-carrying tubes that are configured to transport coolants to various modules of the opto-fluidic instrument for regulating the temperatures thereof. In such cases, the fluidics module may include coolant reservoirs for storing the coolants and pumps (e.g., “coolant pumps”) for generating a pressure differential, thereby forcing the coolants to flow from the reservoirs to the various modules of the opto-fluidic instrument via the coolant-carrying tubes. In some instances, the fluidics module may include returning coolant reservoirs that may be configured to receive and store returning coolants, e.g., heated coolants flowing back into the returning coolant reservoirs after absorbing heat discharged by the various modules of the opto-fluidic instrument. In such cases, the fluidics module may also include cooling fans that are configured to force air (e.g., cool and/or ambient air) into the returning coolant reservoirs to cool the heated coolants stored therein. In some instances, the fluidics module may also include cooling fans that are configured to force air directly into a component of the opto-fluidic instrument so as to cool said component. For example, the fluidics module may include cooling fans that are configured to direct cool or ambient air into the system controller to cool the same.

As discussed above, the opto-fluidic instrument may include an optics module which include the various optical components of the opto-fluidic instrument, such as but not limited to a camera, an illumination module (e.g., LEDs), an objective lens, and/or the like. The optics module may include a fluorescence imaging system that is configured to image the fluorescence emitted by the probes (e.g., oligonucleotides) in the sample after the probes are excited by light from the illumination module of the optics module.

In some instances, the optics module may also include an optical frame onto which the camera, the illumination module, and/or the X-Y stage of the sample module may be mounted.

In various embodiments, the system controller may be configured to control the operations of the opto-fluidic instrument (e.g., and the operations of one or more modules thereof). In some instances, the system controller may take various forms, including a processor, a single computer (or computer system), or multiple computers in communication with each other. In various embodiments, the system controller may be communicatively coupled with data storage, set of input devices, display system, or a combination thereof. In some cases, some or all of these components may be considered to be part of or otherwise integrated with the system controller, may be separate components in communication with each other, or may be integrated together. In other examples, the system controller can be, or may be in communication with, a cloud computing platform.

In various embodiments, the opto-fluidic instrument may analyze the sample and may generate the output that includes indications of the presence of the target molecules (e.g., target RNAs), the presence of which can be indicated by detecting sequences in generated extension product, e.g., an extended nucleic acid molecule or an extended concatemer, in the sample. For instance, with respect to the example embodiment discussed above where the opto-fluidic instrument employs a hybridization technique for detecting generated extension products, e.g., an extended nucleic acid molecule or an extended concatemer, the opto-fluidic instrument may cause the sample to undergo successive rounds of detectably labeled probe hybridization (e.g., using two or more sets of fluorescent probes, where each set of fluorescent probes is excited by a different color channel) and be imaged to detect target molecules in the probed sample. In such cases, the output may include optical signatures (e.g., a codeword) specific to each gene, which allow the identification of the target RNAs.

### VI. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide” and “nucleic acid molecule,” used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

A “primer” as used herein, in some embodiments, is an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

In some instances, “ligation” refers to the formation of a covalent bond or linkage between the termini of two or more nucleic acids, e.g., oligonucleotides and/or polynucleotides, in a template-driven reaction. The nature of the bond or linkage may vary widely and the ligation, in some embodiments, is carried out enzymatically or chemically. As used herein, ligations are usually carried out enzymatically to form a phosphodiester linkage between a 5′ carbon terminal nucleotide of one oligonucleotide with a 3′ carbon of another nucleotide.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of operations in the claims. Similarly, the use of these terms in the specification does not by itself connote any required priority, precedence, or order.

### EXAMPLES

The following examples are included for illustrative purposes only and are not intended to limit the scope of the present disclosure.

### Example 1: Target RNA Cleavage and Extension

This example provides an example of a workflow using a nucleic acid oligonucleotide and RNase H to cleave a target RNA prior to extension of the target RNA using an extension probe and additional extension probes to generate an extended concatemer.

Mouse brains are embedded into OCT medium and directly frozen on dry ice, and thereafter stored at −80° C. until usage. Thin sections are cut with a cryostat and sections collected on glass slides (an optically transparent substrate). Sections are shortly fixated in formaldehyde and paraformaldehyde in PBS and washed, after which they are permeabilized (e.g., with methanol). After the permeabilization, slides are washed in PBS.

A plurality of extension probes are designed to hybridize target sequences at the 3′ end of a plurality of target RNAs. Each extension probe comprises a target recognition sequence complementary to a target sequence at the 3′ end of the target RNA, and a detection sequence. To generate target RNAs that have the target sequence positioned at the 3′ end, the target RNAs are cleaved at specific positions using a single-stranded DNA oligonucleotide complementary to an oligonucleotide hybridization region as illustrated in FIG. 2A. The tissue section are contacted with 1 U RNase H and incubated for 30 minutes at 37° C. in a buffer comprising magnesium chloride, potassium chloride, dithiothreitol (DTT), and Tris-HCL. The tissue section is washed to remove RNase H and DNA oligonucleotide. After the wash, the sample is immersed into a mixture with extension probes and extension is performed using a polymerase and dNTPs. In some cases, prior to polymerization, the sample is treated with T4 PNK to polish ends (e.g., to repair any existing cuts or nicks in the mRNA). An example of extension probe sequences are provided in the middle column of Table 1 and Table 2 for a list of mouse brain target RNAs. In this example, each extension probe has a stem loop structure and the target recognition sequence is underlined. Each stem comprises a detection sequence (e.g., a barcode sequence) associated with the target RNA. In Table 2, a detection sequence (e.g., a barcode sequence) is made up of three nucleotides for improved dissociation and rapid amplification. For example, cytosine (C) is not used in the first stem sequence of the stem loop structure. Such a design is used to reduce the hydrogen bond number and the melting temperature. In some cases, the extension probe and/or additional extension probe comprises a stopper modification or stopper molecule such that polymerization does not incorporate the sequence of the loop into the extension product generated by the polymerase. For example, using the probes in Table 2, the polymerization reaction is performed without guanine in the nucleotide mix to halt polymerization when it approaches the loop sequence (gggccttttggccc (SEQ ID NO: 32)). At the loop, the enzyme may pause or the extension probe may dehybridizing from the target mRNA.

TABLE 1
Probe sequences for mouse brain target RNAs

| Target | | |
|---|---|---|
| RNA | Extension Probe | Additional Extension Probe |
| Myl4 | CACAAGCCTCTCACA (SEQ ID NO: 1) C3 spacer | CACAAGCCTCTCACA (SEQ ID NO: 1) C3 |
| | phosphoramidite gggccttttggccc | spacer phosphoramidite gggccttttggccc |
| | TGTGAGAGGCTTGTG | TGTGAGAGGCTTGTG |
| | AATACTCCGTAACAGTAACAGCCGCTGTGGAT | TGTGAGAGGCTTGTG |
| | /3InvdT/ (SEQ ID NO: 2) | /3InvdT/ (SEQ ID NO: 13) |
| Fmod | CACAGACTACCCACA (SEQ ID NO: 3) C3 spacer | CACAGACTACCCACA (SEQ ID NO: 3) C3 |
| | phosphoramidite gggccttttggccc | spacer phosphoramidite gggccttttggccc |
| | TGTGGGTAGTCTGTG | TGTGGGTAGTCTGTG |
| | TCACTGGTAATCTGGTTGCCATGTAGAGCGAC | TGTGGGTAGTCTGTG /3InvdT/ (SEQ ID NO: |
| | /3InvdT/ (SEQ ID NO: 4) | 14) |
| Gad1 | CACAGCCAATCCACA (SEQ ID NO: 5) C3 spacer | CACAGCCAATCCACA (SEQ ID NO: 5) C3 |
| | phosphoramidite gggccttttggccc | spacer phosphoramidite gggccttttggccc |
| | TGTGGATTGGCTGTG | TGTGGATTGGCTGTG |
| | GATGGCCTAGATGTGTCAGCTACTGACAGAGC | TGTGGATTGGCTGTG /3InvdT/ (SEQ ID NO: |
| | /3InvdT/ (SEQ ID NO: 6) | 15) |
| Ccn2 | CACATCGCTCTCACA (SEQ ID NO: 7) C3 spacer | CACATCGCTCTCACA (SEQ ID NO: 7) C3 |
| | phosphoramidite gggccttttggccc | spacer phosphoramidite gggccttttggccc |
| | TGTGAGAGCGATGTG | TGTGAGAGCGATGTG |
| | TACAGAAGAAAATGAGATGCAACTCAGTTCAA | TGTGAGAGCGATGTG /3InvdT/ (SEQ ID |
| | /3InvdT/ (SEQ ID NO: 8) | NO: 16) |
| Kcnmb2 | CACATTGCCACCACA (SEQ ID NO: 9) C3 spacer | CACATTGCCACCACA (SEQ ID NO: 9) C3 |
| | phosphoramidite gggccttttggccc | spacer phosphoramidite gggccttttggccc |
| | TGTGGTGGCAATGTG | TGTGGTGGCAATGTG |
| | ATAACAACCAAAAGGGAACAGTGAGTAGAAAA | TGTGGTGGCAATGTG /3InvdT/ (SEQ ID |
| | /3InvdT/ (SEQ ID NO: 10) | NO: 17) |
| Rab3b | CACATCAAGCCCACA (SEQ ID NO: 11) C3 spacer | CACATCAAGCCCACA (SEQ ID NO: 11) C3 |
| | phosphoramidite gggccttttggccc | spacer phosphoramidite gggccttttggccc |
| | TGTGGGCTTGATGTG | TGTGGGCTTGATGTG |
| | TCTCTATCTTGAACCTTCTTCATAAGAGGGAG | TGTGGGCTTGATGTG /3InvdT/ (SEQ ID NO: |
| | /3InvdT/ (SEQ ID NO: 12) | 18) |

TABLE 2
Probe sequences for mouse brain target RNAs

| Target | | |
|---|---|---|
| RNA | Extension Probe | Additional Extension Probe |
| Myl4 | ACATCATCAT gggccttttggccc ATGATGATGT | ACATCATCAT gggccttttggccc ATGATGATGT |
| | AATACTCCGTAACAGTAACAGCCGCTGTGGAT | ATGATGATGT /3InvdT/ (SEQ ID NO: 25) |
| | /3InvdT/ (SEQ ID NO: 19) | |
| Fmod | ACCAATAATA gggccttttggccc TATTATTGGT | ACCAATAATA gggccttttggccc TATTATTGGT |
| | TCACTGGTAATCTGGTTGCCATGTAGAGCGAC | TATTATTGGT /3InvdT/ (SEQ ID NO: 26) |
| | /3InvdT/ (SEQ ID NO: 20) | |
| Gad1 | AATAAACCTA gggccttttggccc TAGGTTTATT | AATAAACCTA gggccttttggccc TAGGTTTATT |
| | GATGGCCTAGATGTGTCAGCTACTGACAGAGC | TAGGTTTATT /3InvdT/ (SEQ ID NO: 27) |
| | /3InvdT/ (SEQ ID NO: 21) | |
| Ccn2 | AAATACTCTC gggccttttggccc GAGAGTATTT | AAATACTCTC gggccttttggccc |
| | TACAGAAGAAAATGAGATGCAACTCAGTTCAA | GAGAGTATTT GAGAGTATTT /3InvdT/ |
| | /3InvdT/ (SEQ ID NO: 22) | (SEQ ID NO: 28) |
| Kcnmb2 | ATTATTCACT gggccttttggccc AGTGAATAAT | ATTATTCACT gggccttttggccc AGTGAATAAT |
| | ATAACAACCAAAAGGGAACAGTGAGTAGAAAA | AGTGAATAAT /3InvdT/ (SEQ ID NO: 29) |
| | /3InvdT/ (SEQ ID NO: 23) | |
| Rab3b | ACTTTTTTTC gggccttttggccc GAAAAAAAGT | ACTTTTTTTC gggccttttggccc |
| | TCTCTATCTTGAACCTTCTTCATAAGAGGGAG | GAAAAAAAGT GAAAAAAAGT /3InvdT/ |
| | /3InvdT/ (SEQ ID NO: 24) | (SEQ ID NO: 30) |

After extension with the extension probes, a wash is performed. To further add amplification sequences to the extended nucleic acid molecule comprising the cleaved target RNA, the sample is incubated with additional extension probes, polymerase, and dNTPs to perform further extension. Examples of additional extension probes are provided in the right column of Table 1 and Table 2. After multiple cycles of binding additional extension probes and performing extension using polymerase enzymes, the generated extended concatemer comprises the cleaved target RNA, the detection sequence or complements thereof and multiple copies of the amplification sequence or complements thereof. The generated extended concatemers are then detected in the biological sample by hybridization of detectably labeled probes and imaging the biological sample. In this manner, the generated extended concatemers are covalently bound to the target RNA and this provides positional stability for the sequences in the generated extended concatemers to be detected. In some aspects, the processing of the target RNA (e.g., cleavage), extension reactions, and detection of generated sequences are performed using an automated instrument with integrated optics and fluidics modules (e.g., as described in Section V).

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1.-161. (canceled)

162. A method of nucleic acid processing, comprising:
(a) hybridizing an extension probe to a target ribonucleic acid (RNA), wherein the extension probe comprises i) a target recognition sequence complementary to a target sequence at the 3′ end of the target RNA, and ii) a detection sequence;
(b) extending the target RNA with a polymerase using the extension probe as a template, thereby generating an extended nucleic acid molecule; and
(c) performing further extension of the extended nucleic acid molecule using a plurality of additional extension probes, wherein an additional extension probe of the plurality of additional extension probe comprises (i) at least a portion of the detection sequence of the extension probe or a complement thereof, and (ii) an amplification sequence;
thereby generating an extended concatemer comprising the target RNA, the detection sequence or a complement thereof and the amplification sequence or a complement thereof.

163. The method of claim 162, wherein the amplification sequence is the same sequence as the detection sequence.

164. The method of claim 162, wherein the amplification sequence is different from the detection sequence.

165. The method of claim 162, wherein the extension probe or the additional extension probe comprises a stem loop structure.

166. The method of claim 165, wherein the extension probe or the additional extension probe comprises a stopper molecule or a stopper modification.

167. The method of claim 166, wherein the stopper molecule or stopper modification comprises a chemical modification.

168. The method of claim 166, wherein the stopper molecule is a first nucleotide and the extension or further extension is performed with a plurality of free nucleotides that lack a free nucleotide that base-pairs with the first nucleotide.

169. The method of claim 162, wherein the extension probe comprises at least two different detection sequences or the additional extension probe comprises at least two different amplification sequences.

170. The method of claim 162, wherein the extended concatemer comprises a cleavage site recognized by a restriction enzyme or a DNAzyme.

171. The method of claim 162, wherein the target RNA is a messenger RNA (mRNA).

172. The method of claim 162, wherein the method comprises imaging the extended concatemer.

173. The method of claim 172, wherein the imaging comprises detecting a signal associated with a detectably labeled probe that directly or indirectly binds to the extended concatemer.

174. The method of claim 162, wherein a sequence of the extended concatemer is analyzed by sequential hybridization, sequencing by hybridization, sequencing by ligation, sequencing-by-synthesis (SBS), sequencing-by-avidity (SBA), sequencing-by-binding (SBB), or a combination thereof.

175. The method of claim 174, wherein the sequence of the extended concatemer comprises one or more barcode sequences or complements thereof.

176. The method of claim 175, wherein each barcode sequence or complement thereof of the one or more barcode sequences or complements thereof is assigned a series of signal codes that identifies the barcode sequence or complement thereof, and
wherein detecting the one or more barcode sequences or complements thereof comprises decoding the one or more barcode sequences of complements thereof by detecting the corresponding sequences of signal codes detected from sequential hybridization, detection, and removal of sequential pools of intermediate probes and a universal pool of detectably labeled probes.

177. The method of claim 176, wherein the series of signal codes comprise fluorophore sequences assigned to the corresponding barcode sequences or complements thereof.

178. The method of claim 176, wherein the detectably labeled probes are fluorescently labeled.

179. The method of claim 162, wherein the target RNA is in a biological sample.

180. The method of claim 179, wherein the extended concatemer is analyzed at a location in the biological sample or a matrix embedding the biological sample.

181. The method of claim 179, wherein the biological sample is a cell or tissue sample.
